(19) United States
(12) Patent Application Publication
(10) Pub. No.: US 2004/0192730 A1
(43) Pub. Date: Sep. 30, 2004

(54) Methods of using compounds with combined 5-HT1A and SSRI activities as-needed to treat sexual dysfunction

(75) Inventor: Karl Bruce Thor, Morrisville, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Dynogen Pharmaceuticals, Inc., Boston, MA (US)

(21) Appl. No.: 10/799,490

(22) Filed: Mar. 12, 2004

Related U.S. Application Data
(60) Provisional application No. 60/454,220, filed on Mar. 13, 2003.
(60) Provisional application No. 60/480,596, filed on Jun. 20, 2003.
(60) Provisional application No. 60/545,269, filed on Feb. 17, 2004.

(51) Int. Cl. A61K 31/4545; A61K 31/506; A61K 31/501

(52) U.S. Cl. 514/316; 514/318; 514/252.03; 514/256; 514/314

(57) ABSTRACT

A method is provided for using compounds that exhibit combined activities as 5-HT_{1A} active agents and selective serotonin reuptake inhibitors (SSRI's) to treat sexual dysfunction, particularly premature ejaculation, on an as-needed basis shortly before sexual activity.

### CROSS-REFERENCE TO RELATED APPLICATIONS

This application claims the benefit of U.S. Provisional Application No. 60/454,220, filed Mar. 13, 2003; U.S. Provisional Application No. 60/480,596, filed May 20, 2003; and U.S. Provisional Application No. 60/545,269, filed Feb. 17, 2004 all of which are hereby incorporated by reference.

### FIELD OF THE INVENTION

The invention relates to methods of using compounds that exhibit combined activities as 5-HT_{1A} active agents and selective serotonin reuptake inhibitors (SSRI's) to treat sexual dysfunction, particularly premature ejaculation, on an as-needed basis shortly before sexual activity.

### BACKGROUND OF THE INVENTION

Survey results indicate that sexual dysfunction affects 43 percent of women and 31 percent of men in the US. (Laumann et al. (1999) JAMA, 281: 537-44). Although sexual dysfunction may take a variety of forms, the term generally refers to a disturbance of normal human sexual response.

Premature ejaculation (PE) is one of the most common sexual complaints and has been estimated to affect up to 30 to 40 percent of American men (Derogatis, L. R., Med. Aspects Hum. Sexuality, 14: 1168-76 (1980); Frank E., et al., N. Engl. J. Med., 299: 111-115 (1978); Schein, M., et al., Fam. Pract. Res. J., 7 (3): 122-134 (1988)). PE is typically characterized as persistent or recurrent ejaculation with minimal sexual stimulation before, upon, or shortly after penetration, and before the person wishes it. Because of associated emotional and psychological repercussions, PE often leads to other sexual dysfunctions including male erectile dysfunction (MED), female sexual dysfunction (FSD) including anorgasmia and hypoactive sexual desire, and sexual aversion. (Rust et al. (1988) Br. J. Psychiat., 152: 629-631). Causes of PE maybe psychological or may involve prostate gland inflammation or nervous system disorders.

The current standard of therapy for treatment of PE is behavioral therapy, including such approaches as the Semans pause maneuver, the Masters and Johnson pause-squeeze technique or the Kaplan stop-start method (Seftel, A. D., Altohob, S. E., “Premature Ejaculation”, Diagnosis and Management of Male Sexual Dysfunction, Edited by J. J. Mulcahy, New York, N.Y., Igaku-Shoin, (1997) Chapter 11, pages 196-203). Although these techniques may be successful at rates of 60 to 95% (Seftel, supra; Hawton, K., et al., Behav. Res. Ther., 24: 377 (1986)), they require partner cooperation and improvement is usually short-lived (Bancroft, J. and Coles, L., Brit. Med. J., 1: 1575 (1976) and De Amicus, L. A., et al., Arch. Sex. Behav., 14: 467 (1985)). Drug treatment options include using tricyclic antidepressants or certain selective serotonin re-uptake inhibitor drugs (Merck Manual of Medical Information at 421-422, Home Edition, Merck Research Laboratories (1997)); see also U.S. Pat. No. 5,597,826 (sertraline), U.S. Pat. No. 5,276,042 (paroxetine), and U.S. Pat. No. 5,151,448 (fluoxetine). However, these treatments primarily concern the chronic administration of therapeutic agents for the treatment of PE, and some of these treatments produce undesirable side effects, including MED.

Because existing therapies and treatments for sexual dysfunction are associated with limitations as described above, new therapies and treatments for sexual dysfunction are therefore desirable.

### SUMMARY OF THE INVENTION

Compositions and methods for treating sexual dysfunction, particularly PE, are provided. Compositions of the invention comprise compounds exhibiting combined 5-HT_{1A} receptor and SSRI activities formulated for administration on an as-needed basis, as well as pharmaceutically acceptable, pharmacologically active salts, enantiomers, analogs, esters, amides, prodrugs, metabolites, and derivatives of said compounds. The compositions are formulated and administered in therapeutically effective amounts on an as-needed basis to a patient in need thereof for treating sexual dysfunction, particularly premature ejaculation (PE).

### DETAILED DESCRIPTION OF THE INVENTION

Overview and Definitions

The present invention provides compositions and methods for treating sexual dysfunction, particularly PE. The compositions comprise a therapeutically effective dose of a compound exhibiting combined 5-HT_{1A} receptor and SSRI activities formulated for administration on an as-needed basis, as well as pharmaceutically acceptable, pharmacologically active salts, esters, amides, prodrugs, active metabolites, and other derivatives of said compound. The methods are accomplished by administering on an as-needed basis various compositions and formulations containing a compound exhibiting combined 5-HT_{1A} receptor and SSRI activities.

Before describing the present invention in detail, it is to be understood that this invention is not limited to specific active agents, dosage forms, dosing regimens, or the like, as long as the invention may be used on an as-needed basis. It is also to be understood that the terminology used herein is for the purpose of describing particular embodiments only, and is not intended to be limiting.

It must be noted that as used in this specification and the appended embodiments, the singular forms “a,” an” and “the” include plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

“Sexual dysfunction” is used in its conventional sense to refer to any disturbance of normal human sexual response. Disorders of sexual dysfunction include, but are not limited to, premature ejaculation.

“Normal human sexual response” refers to sexual response that includes three stages including a desire stage, an arousal stage, and an orgasm stage as described in Helen Singer Kaplan (1979) Disorders of Sexual Desire, Brunner Mazel Book, Inc., New York, N.Y.

“Premature ejaculation” (or “PE”) is used in its conventional sense to refer to persistent or recurrent ejaculation with minimal sexual stimulation before, upon, or shortly after penetration, and before the person wishes it. “Rapid ejaculation” is synonymous with premature ejaculation and is used frequently in the literature.

As used herein the term “sexual activity” refers to an activity involving sexual arousal wherein the patient desires to avoid sexual dysfumction including PE. Examples of sexual activity are masturbation, sexual intercourse, intromission, and the like, although sexual intercourse is preferred. As used herein the term “sexual intercourse” refers to physical stimulation between individuals that involves the genitalia of at least one person, such as intromission. As used herein the term “intromission” refers to the insertion or period of insertion of the penis into an orifice. An example of an orifice is the vagina. As used herein the term “sexual arousal” refers to engorgement of a sexual organ. Examples of sexual organs are the penis and clitoris.

As used herein the term “engorgement” refers to an increase in blood flow to a sexual organ.

The terms “active agent” and “pharmacologically active agent” are used interchangeably herein to refer to a chemical compound that induces a desired effect, i.e., in this case, treatment of sexual dysfunction, particularly PE. The primary active agents herein are compounds exhibiting combined 5-HT_{1A} receptor and SSRI activities formulated for administration on an as-needed basis. Such formulations include the use of an active agent that is a rapid-onset compound exhibiting combined 5-HT_{1A} receptor and SSRI activities, as described herein. Such formulations also include the use of an active agent that is a short acting compound exhibiting combined 5-HT_{1A} receptor and SSRI activities, as described herein. In addition, active agents may include compounds exhibiting combined 5-HT_{1A} receptor and SSRI activities that are both rapid-onset and short acting, as described herein. In addition, the formulation may also include agents that are long-acting but can be administered at an initial dose that achieves therapeutic plasma concentrations rapidly and the administration of that dose can be safely repeated as often as the patient desires. Finally, a combination therapy wherein a compound exhibiting combined 5-HT_{1A} receptor and SSRI activities formulated for administration on an as-needed basis is administered with one or more additional active agents is also within the scope of the present invention. Such combination therapy may be carried out by administration of the different active agents in a single composition, by concurrent administration of the different active agents in different compositions, or by sequential administration of the different active agents. Included are salts, enantiomers, analogs, esters, amides, prodrugs, active metabolites, and derivatives of those compounds or classes of compounds specifically mentioned that also induce the desired effect.

The term “compound exhibiting combined 5-HT_{1A} receptor and SSRI activities” as used herein is intended to refer to a single agent that is both an SSRI and also acts as an antagonist or partial agonist of the 5-HT_{1A} receptor. Unless otherwise indicated, the term “compound exhibiting combined 5-HT_{1A} receptor and SSRI activities” refers to a single agent that is both an SSRI and also acts as an antagonist or partial agonist ofthe 5-HT_{1A} receptor as disclosed further herein, as well as salts, enantiomers, analogs, esters, amides, prodrugs, active metabolites, and other derivatives thereof. Further, it is understood that any salts, enantiomers, analogs, esters, amides, prodrugs, active metabolites, or other derivatives are pharmaceutically acceptable as well as pharmacologically active. Furthermore, as used herein, the term “5-HT_{1A} active agents” includes compounds that act as antagonists or partial agonists of the 5-HT_{1A} receptor and the term “5-HT_{1A} activity” refers to antagonism or partial agonism of the 5-HT_{1A} receptor.

As used herein the term “partial agonist” refers to a drug that interacts with the receptor, including a binding event, and produces a degree of activation of the receptor that is consistently equal to or less than about 75% of the degree of activation of the receptor by endogenous neurotransmitter 5-hydroxytryptamine (5-HT), about 70% of the degree of activation of the receptor by endogenous 5-HT, about 65% of the degree of activation of the receptor by endogenous 5-HT, about 60% of the degree of activation of the receptor by endogenous 5-HT, about 55% of the degree of activation of the receptor by endogenous 5-HT, about 50% of the degree of activation of the receptor by endogenous 5-HT, about 45% of the degree of activation of the receptor by endogenous 5-HT, about 40% of the degree of activation of the receptor by endogenous 5-HT, about 35% of the degree of activation of the receptor by endogenous 5-HT, about 30% of the degree of activation of the receptor by endogenous 5-HT, about 25% of the degree of activation of the receptor by endogenous 5-HT, about 20% of the degree of activation of the receptor by endogenous 5-HT, about 15% of the degree of activation of the receptor by endogenous 5-HT, about 10% of the degree of activation of the receptor by endogenous 5-HT, about 5% of the degree of activation of the receptor by endogenous 5-HT, about 4% of the degree of activation of the receptor by endogenous 5-HT, about 3% of the degree of activation of the receptor by endogenous 5-HT, about 2% of the degree of activation of the receptor by endogenous 5-HT, about 1% of the degree of activation of the receptor by endogenous 5-HT, or about 0.5% of the degree of activation of the receptor by endogenous 5-HT. In addition, while a partial agonist is bound to the receptor, the partial agonist prevents the binding of endogenous transmitter 5-HT.

As used herein the term “antagonist” refers to a drug that interacts with the receptor, including a binding event, but does not measurably activate the receptor and prevents the binding of the endogenous neurotransmitter 5-hydroxytryptamine (5-HT).

As used herein the term “a rapid-onset compound exhibiting combined 5-HT_{1A} receptor and SSRI activities” or “rapid-onset” refers to a drug with a pharmacokinetic profile wherein Tₘₐₓ (time to peak plasma levels) is consistently less than about 4 hours, consistently less than about 3 hours, consistently less than about 2 hours, consistently less than about 1 hour, or consistently less than about 30 minutes.

As used herein the term “a short acting compound exhibiting combined 5-HT_{1A} receptor and SSRI activities” or “short-acting” refers to a drug with a pharmacokinetic profile wherein T_{1/2} (plasma elimination half-life) is less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, or less than about 1 hour.

As used herein, the term “agents that are long-acting but can be administered at an initial dose that achieves therapeutic plasma concentrations rapidly and the administration of that dose can be safely repeated as often as the patient desires” refers to a rapid-onset compound exhibiting combined 5-HT_{1A} receptor and SSRI activities that has a T_{1/2} (plasma elimination half-life) that produces accumulation of drug in the plasma if the drug is taken repeatedly or daily, but the accumulation of the drug does not produce intolerable side-effects (particularly, where 5 times the therapeutic plasma concentration is not toxic).

The terms “treating” and “treatment” as used herein refer to relieving the symptoms associated with sexual dysfumction, particularly PE.

By an “effective” amount or a “therapeutically effective amount” of a drug or pharmacologically active agent is meant a nontoxic but sufficient amount of the drug or agent to provide the desired effect, i.e., relieving the symptoms associated with sexual dysfuction, particularly PE, as explained above. It is recognized that the effective amount of a drug or pharmacologically active agent will vary depending on the route of administration, the selected compound, and the species to which the drug or pharmacologically active agent is administered. It is also recognized that one of skill in the art will determine appropriate effective amounts by taking into account such factors as metabolism, bioavailability, and other factors that affect plasma levels of a drug or pharmacologically active agent following administration within the unit dose ranges disclosed further herein for different routes of administration.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound. When the term “pharmaceutically acceptable” is used to refer to salts, enantiomers, analogs, esters, arnides, prodrugs, active metabolites, and derivatives of an active agent, it is to be understood that the compound is pharmacologically active as well, i.e., therapeutically effective for treating PE.

By “as-needed” dosing, also known as “pro re nata” “pm” dosing, and “on demand” dosing or administration is meant the administration of a single dose of the active agent at some time prior to commencement of an activity wherein suppression of the symptoms of PE, would be desirable. Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

By “short-term” is intended any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

By “rapid-offset” is intended any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “non-immediate release” as defined in Remington: The Science and Practice of Pharmacy, Twentieth Ed. (Philadelphia, Pa.: Lippincott Williams & Wilkins, 2000).

The “absorption pool” represents a solution of the drug administered at a particular absorption site, and kᵣ, kₐ, and kₑ are first-order rate constants for: 1) release of the drug from the formulation; 2) absorption; and 3) elimination, respectively. For immediate release dosage forms, the rate constant for drug release kᵣ is far greater than the absorption rate constant kₐ. For controlled release formulations, the opposite is true, i.e., kᵣ<<<kₐ, such that the rate of release of drug from the dosage form is the rate-limiting step in the delivery of the drug to the target area. The term “controlled release” as used herein includes any nonimmediate release formulation, including but not limited to sustained release, delayed release and pulsatile release formulations.

The term “sustained release” is used in its conventional sense to refer to a drug formulation that provides for gradual release of a drug over an extended period of time, and that preferably, although not necessarily, results in substantially constant blood levels of a drug over an extended time period. One of skill in the art would readily recognize that sustained release formulations of the present invention must be formulated to allow for as-needed administration, as defined further herein.

The term “delayed release” is used in its conventional sense to refer to a drug formulation that provides for an initial release of the drug after some delay following drug administration. One of skill in the art would readily recognize that delayed release formulations of the present invention must be formulated to allow for as-needed administration, as defined further herein.

The term “pulsatile release” is used in its conventional sense to refer to a drug formulation that provides release of the drug in such a way as to produce pulsed plasma profiles of the drug after drug administration. One of skill in the art would readily recognize that pulsatile release formulations of the present invention must be formulated to allow for as-needed administration, as defined further herein.

By the term “transdermal” drug delivery is meant delivery by passage of a drug through the skin or mucosal tissue and into the bloodstream.

The term “topical administration” is used in its conventional sense to mean delivery of a topical drug or pharmacologically active agent to the skin or mucosa.

The term “oral administration” is used in its conventional sense to mean delivery of a drug through the mouth and ingestion through the stomach and digestive tract.

The term “inhalation administration” is used in its conventional sense to mean delivery of an aerosolized form of the drug by passage through the nose or mouth during inhalation and passage of the drug through the walls of the lungs.

By the term “parenteral” drug delivery is meant delivery by passage of a drug into the blood stream without first having to pass through the alimentary canal, or digestive tract. Parenteral drug delivery may be “subcutaneous,” referring to delivery of a drug by administration under the skin. Another form of parenteral drug delivery is “intramuscular,” referring to delivery of a drug by administration into muscle tissue. Another form of parenteral drug delivery is “intradermal,” referring to delivery of a drug by administration into the skin. An additional form of parenteral drug delivery is “intravenous,” referring to delivery of a drug by administration into a vein. An additional form of parenteral drug delivery is “intra-arterial,” referring to delivery of a drug by administration into an artery. Another form of parenteral drug delivery is “transdermal,” referring to delivery of a drug by passage of the drug through the skin and into the bloodstream.

Still another form of parenteral drug delivery is “transmucosal,” referring to administration of a drug to the mucosal surface of an individual so that the drug passes through the mucosal tissue and into the individual's blood stream. Transmucosal drug delivery may be “buccal” or “transbuccal,” referring to delivery of a drug by passage through an individual's buccal mucosa and into the bloodstream. Another form of transmucosal drug delivery herein is “lingual” drug delivery, which refers to delivery of a drug by passage of a drug through an individual's lingual mucosa and into the bloodstream. Another form of transmucosal drug delivery herein is “sublingual” drug delivery, which refers to delivery of a drug by passage of a drug through an individual's sublingual mucosa and into the bloodstream. Another form of transmucosal drug delivery is “nasal” or “intranasal” drug delivery, referring to delivery of a drug through an individual's nasal mucosa and into the bloodstream. An additional form of transmucosal drug delivery herein is “rectal” or “transrectal” drug delivery, referring to delivery of a drug by passage of a drug through an individual's rectal mucosa and into the bloodstream. Another form of transmucosal drug delivery is “urethral” or “transurethral” delivery, referring to delivery of the drug into the urethra such that the drug contacts and passes through the wall of the urethra.

“Rapidly disintegrating tablet” as used herein, refers to tablets or wafers that can disintegrate within up to 30 seconds of being placed in the presence of water. Rapidly disintegrating tablets include, but are not limited to, effervescent tablets or wafers and open matrix network tablets. “Effervescent tablets” or “effervescent wafers” as used herein refers to tablets as described in Remington, supra, and U.S. Pat. No. 5,211,957 that generally contain an active agent in combination with additives that react with water to liberate carbon dioxide, thereby facilitating the disintegration of the tablet. Once the tablet is substantially disintegrated, an individual swallows the resultant solution thereby providing systemic adsorption of the active agent. “Open matrix network tablets” as used herein refers to tablets as described in U.S. Pat. No. 4,371,516 that can disintegrate within five to ten seconds after being placed on the tongue of an individual, allowing the contents of the tablet can then be swallowed with or without water. Other examples of rapidly disintegrating tablets that can be adapted to contain active agents as disclosed herein are well-known in the art, including those as discloses in U.S. Pat. No. 5,776,492.

In order to carry out the method of the invention, a selected active agent is administered to a patient diagnosed with sexual dysfunction, particularly PE. A therapeutically effective amount of the active agent may be administered orally, transmucosally (including buccally, sublingually, transurethrally, and rectally), topically, transdermally, by inhalation, or using any other route of administration, so long as the active agent is formulated for as-needed administration.

Premature Ejaculation

A normal erection occurs as a result of a coordinated vascular event in the penis, which is usually triggered neurally and includes vasodilation and smooth muscle relaxation in the penis and its supplying arterial vessels. Arterial inflow causes enlargement of the substance of the corpora cavemosa. Venous outflow is trapped by this enlargement, permitting sustained high blood pressures in the penis normally sufficient to cause rigidity. Muscles in the perineum also assist in creating and maintaining penile rigidity. Erection may also be induced centrally in the nervous system by sexual thoughts or fantasy, and is usually reinforced locally by reflex mechanisms. Erectile mechanics in women are substantially similar for the clitoris. In men, however, ejaculation typically occurs with an orgasm.

Survey results indicate that sexual dysfunction affects 43 percent of women and 31 percent of men in the US. (Laumann et al. (1999) JAMA, 281: 537-44). Although sexual dysfumction may take a variety of forms, the term generally refers to a disturbance of normal human sexual response.

Premature ejaculation (PE) is one of the most common sexual complaints and is estimated to affect up to 30 to 40 percent of American men (Derogatis, L. R., Med. Aspects Hum. Sexuality, 14: 1168-76 (1980); Frank E., et al., N. Engl. J. Med., 299: 111-115 (1978); Schein, M., et al., Fam. Pract. Res. J., 7 (3): 122-134 (1988)). PE is typically characterized as persistent or recurrent ejaculation with minimal sexual stimulation before, upon, or shortly after penetration, and before the person wishes it. Because of associated emotional and psychological repercussions, PE often leads to other sexual dysfunctions including male erectile dysfunction (MED), female sexual dysfumction (FSD) including anorgasmia and hypoactive sexual desire, and sexual aversion. (Rust et al. (1988) Br. J. Psychiat., 152: 629-631). Causes of PE may be psychological or may involve prostate gland inflammation or nervous system disorders.

Although ejaculation latency is affected by psychological and/or cognitive mechanisms, somatic factors are also involved (Althof, S. E. (1995) Psychiatr. Clin. N. Amer., 18: 85-94; Rowland, D. L., et al., (1993) J. Sex. Marital. Ther., 19: 189). Ejaculation is mediated partly through a neural reflex stimulated by sensory input to the penis, and terminating in smooth and striated muscle contractions that produce seminal emission and expulsion. Segraves hypothesized that increased serotoninergic activation may be associated with orgasmic inhibition (Arch. Gen. Psychiatry., 46: 275-284 (1989)) and reports that ejaculation seems to be mediated by al adrenergic receptor activation, presumably at a peripheral level with cholinergic fibers playing a modulatory role. Serotoninergic system involvement in ejaculation could occur at the level of the brain or spinal cord.

The current standard of therapy for treatment of PE is behavioral therapy, including such approaches as the Semans pause maneuver, the Masters and Johnson pause-squeeze technique or the Kaplan stop-start method (Seftel, A. D., Altohob, S. E., “Premature Ejaculation”, Diagnosis and Management of Male Sexual Dysfunction, Edited by J. J. Mulcahy, New York, N.Y., Igaku-Shoin, (1997) Chapter 11, pages 196-203). Although these techniques may be successful at rates of 60 to 95% (Seftel, supra; Hawton, K., et al., Behav. Res. Ther., 24: 377 (1986)), they require partner cooperation and improvement is usually short-lived (Bancroft, J. and Coles, L., Brit. Med. J., 1: 1575 (1976) and De Amicus, L. A., et al., Arch. Sex. Behav., 14: 467 (1985)). Drug treatment options include using tricyclic antidepressants or certain selective serotonin re-uptake inhibitor drugs (Merck Manual of Medical Information at 421-422, Home Edition, Merck Research Laboratories (1997)); see also U.S. Pat. No. 5,597,826 (sertraline), U.S. Pat. No. 5,276,042 (paroxetine), and U.S. Pat. No. 5,151,448 (fluoxetine). However, these treatments primarily concern the chronic administration of therapeutic agents for the treatment of PE, and some of these treatments produce undesirable side effects, including MED.

Proper treatment of PE involves not just inhibiting early ejaculation, but in ensuring that the patient has increased control over the timing of the ejaculation. The currently available options for treating PE typically require daily dosage to maintain suitable plasma levels, which may produce undesirable or dangerous side effects. For example, the daily or chronic use of conventional SSRI's and related compounds for such therapy may result in adverse effects expected with high or continuing dosages of such compounds. Chronic or daily administration of conventional SSRI's is also a burdensome requirement on the patient. Furthermore, the latency period, from time of dosing to engaging in sexual activity, associated with conventional SSRI's is another hurdle that the patient must deal with. Finally, not experiencing benefit from a drug with a single, or the first, administration of drug is also burdensome.

Serotonin Receptors

Serotonin (5-hydroxytryptamine or 5-HT) is a neurotransmitter that has been implicated in various psychiatric diseases including depression, obsessive-compulsive disorder, panic disorder, and sexual disorders. (Olivier et al. (1998) Int. Clinical Psychopharm. 13 (suppl. 6): S9-S14). At least 14 different serotonin receptors have been distinguished, including 5-HT_{1A}, 5-HT_{1B}, 5-HT_{1D}, 5-HT_{1E}, 5-HT_{1F}, 5-HT₂A, 5-HT₂B, 5-HT₂C, 5-HT₃, 5-HT₄, 5-HT₅A, 5-HT₅B, 5-HT₆, 5-HT₇. (Olivier et al, (1998) Int. Clinical Psychopharm. 13 (suppl. 6): S9-S14).

The 5-HT system is highly heterogeneous and complex, and the various receptor subtypes possess a variety of distinguishing characterstics including anatomical localization to different parts of the central nervous system and division into two extended gene superfamilies: the G-protein coupled receptor (GPCR) superfamily and the ligand-gated ion channel superfamily. (Olivier et al. (1998) Int. Clinical Psychopharm. 13 (suppl. 6): S9-S14). The 5-HT₁, 5-HT₂, 5-HT₄, 5-HT₅, 5-HT₆, and 5-HT₇ families of receptors are linked to the modulation of either GPCR mediated adenylate cyclase or phosphoinositol turnover and display a seven transmembrane domain, while 5-HT₃ receptors modulate ion channels and consists of 5 subunits. (Olivier et al. (1998) Int. Clinical Psychopharm. 13 (suppl. 6): S9-S14). In addition, subtypes of serotonin receptors may couple to different effector systems depending upon the cell type in which it is expressed, and even when two subtypes are expressed in the same neurons they may be localized in different subcellular areas (e.g., 5-HT_{1A} receptors are found mainly somatodendritically in neurons of the raphe nucleus while 5-HT_{1B} receptors are localized on axon terminals of the same neurons). (Olivier et aL (1998) Int. Clinical Psychopharm. 13 (suppl. 6): S9-S14).

5-HT_{1A} Agonists/Partial Agonists

5-HT_{1A} receptor agonists and partial agonists have been identified as anxiolytic agents, antidepressants, antiemetics, and as neuroprotective agents.

5-HT_{1A} agonists and partial agonists have shown anxiolytic activity in both preclinical animal models of anxiety and in clinical trials for anxiety (Chiaie, et aL (1995) J. Clin. Psychopharamacol., 15: 12; Rogers et al. (1994) Pharmacol. Biochem. Behav., 48: 959; Curle et al. (1994) Drug Dev. Res. 32: 183; File and Andrews (1994) Behav. Pharmacol. 5: 99; Westenberg and Den Boer (1993) Pharmacopsychiatry, 26: 30; Chaney et al. (1 990) Ann. Rev. Med. 41: 437).

The ability of 5-HT_{1A} agonists and partial agonists to act on post-synaptic receptors while desensitizing pre-synaptic autoreceptors is though to underlie their antidepressant effects. Such agents identified as antidepressants include buspirone and flesinoxan (Sambunaris et al. (1997) J. Clin. Psychiatry, 58: 40; De Vry (1996) Drug and News Perspectives, 9, 270).

Animal studies have demonstrated that 5-HT_{1A} agonists are effective antiemetics as shown using motion sickness and xylazine- and cis-platinin-induced vomiting models (Lucot (1994) Eur. J. Pharmacol., 253: 53; Lucot and Crampton (1989) Pharmacol. Biochem. Behav., 33: 627).

5-HT_{1A} receptors are located in brain areas that are highly sensitive to ischemia, such as the hippocampus and cerebral cortex. Furthermore, it has been demonstrated that 5-HT_{1A} agonists and partial agonists are able to attenuate glutamate release, most likely through activation of 5-HT_{1A} receptors on glutamatergic terminals (Matsuyama et al. (1996) Brain Res. 728: 175). Glutamate is the predominant excitatory neurotransmitter in the central nervous system and has been associated with ischemia-induced pathophysiology seen in neurodegenerative disorders such as stroke, transient ischemic attack, fetal hypoxia and spinal/brain trauma, epilepsy, Alzheimer's Disease, amyotrophic lateral sclerosis, Huntingdon's Disease, Parkinson's Disease, AIDS dementia, and retinal diseases. (Holt et al. (1997) Glutamate in Health and Disease: The Role of Inhibitors, In Neuroprotection in CNS Diseases, Bar and Beal, eds., Marcel Dekker, Inc., New York, pp. 87-119). Therefore, compounds such as 5-HT_{1A} agonists and partial agonists that are able to attenuate glutamate release have been investigated as potential neuroprotective agents.

The identification of 5-HT_{1A} partial agonists can be determined by methods known to one of skill in the art, including measurement of inhibition of forskolin-stimulated accumulation of cAMP in CHO cells stably transfected with the human 5-HT_{1A} receptor. (See, e.g., Dunlop et al. (1998) J. Pharmacol. Tox. Methods, 40: 47-55; Martinez-Esparza et al. (2001) J. Med. Chem. 44:418-428; Tordera et al. (2002) Eur. J. Pharmacol. 442:63-71; Martinez et al. (2001) Eur. J. Med. Chem. 36: 55-61).

In a preferred embodiment of the present invention, the 5-HT_{1A} active agent is a 5-HT_{1A} anatagonist.

In another preferred embodiment of the present invention, the 5-HT_{1A} active agent is a 5-HT_{1A} partial agonist of the 5-HT_{1A} receptor and produces a degree of activation of the receptor that is consistently equal to or less than about 75% of the degree of activation of the receptor by endogenous 5-HT, about 70% of the degree of activation of the receptor by endogenous 5-HT, about 65% of the degree of activation of the receptor by endogenous 5-HT, about 60% of the degree of activation of the receptor by endogenous 5-HT, about 55% of the degree of activation of the receptor by endogenous 5-HT, about 50% of the degree of activation of the receptor by endogenous 5-HT, about 45% of the degree of activation of the receptor by endogenous 5-HT, about 40% of the degree of activation of the receptor by endogenous 5-HT, about 35% of the degree of activation of the receptor by endogenous 5-HT, about 30% of the degree of activation of the receptor by endogenous 5-HT, about 25% of the degree of activation of the receptor by endogenous 5-HT, about 20% of the degree of activation of the receptor by endogenous 5-HT, about 15% of the degree of activation of the receptor by endogenous 5-HT, about 10% of the degree of activation of the receptor by endogenous 5-HT, about 5% of the degree of activation of the receptor by endogenous 5-HT, about 4% of the degree of activation of the receptor by endogenous 5-HT, about 3% of the degree of activation of the receptor by endogenous 5-HT, about 2% of the degree of activation of the receptor by endogenous 5-HT, about 1% of the degree of activation of the receptor by endogenous 5-HT, or about 0.5% of the degree of activation of the receptor by endogenous 5-HT.

Metabolism

Drug elimination is primarily the result of metabolic activity upon the drug followed by the excretion of the drug from the body. Although a principal site of drug metabolism is the liver, metabolic activity can also occur within the vascular supply and/or within cellular compartments or other organs. The metabolic process can be divided into two types of reactions: non-synthetic (Phase I) and synthetic (Phase II).

In non-synthetic reactions, a drug is chemically altered by reactions that include oxidation, reduction, hydrolysis, or any combination thereof. (See, for e.g., Remington: The Science and Practice of Pharmacy, supra). In synthetic reactions, also known as conjugations, a parent drug or its intermediate metabolites are combined with endogenous substrates to yield an addition or conjugation product. Metabolites formed in synthetic reactions are typically more polar and biologically inactive and are more easily excreted in urine via the kidneys or in bile via the liver. Synthetic reactions include glucuronidation, amino acid conjugation, acetylation, sulfoconjugation, and methylation. (See, for e.g., Remington: The Science and Practice of Pharmacy, supra).

In a preferred embodiment of the invention, the types of side effects that occur with individual administration of either a 5-HT_{1A} active agent or an SSRI are reduced or eliminated by selecting an active agent in which metabolism of said active agent results in the loss of both 5-HT_{1A} receptor and SSRI activity for the active agent and/or its metabolite or metabolites. Furthermore, active agents are selected for which their metabolite or metabolites do not interfere with the therapeutic efficacy of the parent drug (i.e. the active agent), nor do they produce deleterious side effects. Preferably, metabolism of the active agent may result in a loss of 5-HT_{1A} receptor activity within about 6 hours of the loss of SSRI activity, within about 5.5 hours of the loss of SSRI activity, within about 5.0 hours of the loss of SSRI activity, within about 4.5 hours of the loss of SSRI activity, within about 4.0 hours of the loss of SSRI activity, within about 3.5 hours of the loss of SSRI activity, within about 3.0 hours of the loss of SSRI activity, within about 2.5 hours of the loss of SSRI activity, within about 2.0 hours of the loss of SSRI activity, within about 1.5 hours of the loss of SSRI activity, within about 1.0 hours of the loss of SSRI activity, within about 50 minutes of the loss of SSRI activity, within about 40 minutes of the loss of SSRI activity, within about 30 minutes of the loss of SSRI activity, within about 20 minutes of the loss of SSRI activity, within about 10 minutes of the loss of SSRI activity within about 5 minutes of the loss of SSRI activity, or at about the same time as the loss of SSRI activity.

Identification of active agents in which metabolism of said active agent results in the loss of both 5-HT_{1A} receptor activity and SSRI activity for the active agent and/or its metabolite or metabolites involves: 1) identifying the metabolite or metabolites of the active agent from plasma and/or tissue samples of animals or human volunteers; 2) synthesizing the identified metabolite or metabolites; and 3) examining the metabolite or metabolites for 5-HT1A receptor and SSRI activities.

Identification of Metabolite(s)

The identification of the metabolite or metabolites of the active agent may be conducted by administering the active agent to an animal or human volunteer via a selected administration route as described further herein, followed by the collection of periodic plasma samples from the animal or human volunteer to be analyzed for the detection of the active agent and its metabolite or metabolites. Analysis of plasma samples for the detection of the active agent and its metabolite or metabolites may include the use of mass spectrometry and chromatographic methods such as high-performance liquid chromatography (HPLC), gas chromatography, stereospecific HPLC, and the like. (See, e.g., Ma et aL (2002) J. Chromatogr. B. Analyt. Technol. Biomed. Life Sci. 780: 99-110; Lundkvist et al. (1996) Nucl. Med. Biol. 23: 627-34; Peyton et al. (1991) Pharm. Res. 8: 1528-32; Zuideveld et al. (2000) J. Chrmatogr. B. Biomed. Sci. Appl. 738: 67-73; Rochat et al. (1995) Ther. Drug Monit. 17: 273-9; Hamilton and Cornpropst (1993) J. Chromatogr. 612:253-261; Bernstein et al. (1994) Biopharm. Drug Dispos. 15:137-150; Baumann (1996) Clin. Pharmacokinet. 31: 444-69; Farde et al. (1998) J. Nucl. Med.39: 1965-71).

In an alternative method, human and/or animal hepatocytes may be utilized to produce the metabolite or metabolites of the active agent in lieu of administration of the active agent to an animal or human volunteer. (See, e.g., Ma et al. (2002) J. Chromatogr. B. Analyt. Technol. Biomed. Life Sci. 780: 99-110).

Synthesis of Metabolite(s)

The synthesis of the metabolite or metabolites identified from metabolism of a selected active agent may be conducted using methods and techniques known to one of skill in the art.

Examination of 5-HT_{1A} Receptor and SSRI Activities

Examination of the metabolite or metabolites for their 5-HT_{1A} receptor and SSRI activities may be conducted using general radioligand binding assays. (See, e.g., Owens et al. (1997) J. Pharmacol. Exp. Ther. 283: 1305-22). For example, radioligand binding assays may be utilized to measure the ability of the metabolite or metabolites to block [³H]-paroxetine binding for SSRI activity or to block [³H]-8-OH-dipropylaminotetraline (8-OH-DPAT) binding for 5-HT_{1A} receptor activity (See, e.g., Marazziti et al. (1998) Neuropsychopharmacology 19: 154-9; Fletcher et al. (1996) Behav. Brain Res. 73: 337-53).

Examination of the metabolite or metabolites for their 5-HT_{1A} receptor and SSRI activities may also be conducted using assays of functional activity. For example, SSRI activity may be characterized by determining 5-HT reuptake inhibition in synaptosomes. (See, e.g., Hatanaka et al. (1996) Neuropharmacology 35: 1621-6; Tordera et al. (2002) Eur. J. Pharmacol. 442:63-71). For 5-HT_{1A} receptor activity, including both antagonism and partial agonism of the 5-HT_{1A} receptor, functional assays include, for example, measuring antagonism to 8-OH-DPAT-induced hypothermia in mice, or assessing the 8-OH-DPAT-induced inhibition of forskolin-stimulated cAMP formation in cell lines expressing the 5-HT_{1A} receptor. (See, e.g., Dunlop et al. (1998) J. Pharmacol. Tox. Methods, 40: 47-55; Martinez-Esparza et al. (2001) J. Med. Chem. 44:418-428; Tordera et al. (2002) Eur. J. Pharmacol. 442:63-71; Martinez et al. (2001) Eur. J. Med. Chem. 36: 55-61).

Agents

Compounds useful in the present invention include any active agent as defined elsewhere herein (namely, a chemical compound that induces a desired effect, i.e., treatment of sexual dysfunction, particularly PE). Such active agents include, for example, compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as defined elsewhere herein. Such active agents also include agents that are long-acting but can be administered at an initial dose that achieves therapeutic plasma concentrations rapidly and the administration of that dose can be safely repeated as often as the patient desires, as defined elsewhere herein. Such agents include, for example:

1. Substituted-benzyl or substituted-indolyl cyclic amino-substituted N-aryl or heteroaryl cyclic amines (illustrated below) as disclosed in U.S. Pat. No. 6,225,324 and salts, enantiomers, analogs, esters, amides, prodrugs, active metabolites, and derivatives thereof;

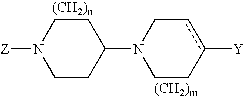

and/or hydrates thereof wherein

Z is selected from phenyl, benzodioxolone, benzodioxole, benzothiazole, pyridine, pyridazine, pyrimidine, and quinoline moieties that are unsubstituted or optimally substituted with one to three substituents selected from C₁₋₄ alkyl, C₁₋₄ alkoxy, cyano, and halo;

the solid and dotted lines denote either a double or a single covalent bond;

m and n are independently integers 1 to 3; and

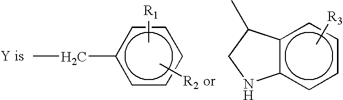

in which R₁ and R₂ are independently selected from hydrogen, halogen, or alkoxy and R₃ is hydrogen, halogen, or cyano, and

particularly including BMS-296859 (illustrated below) and salts, enantiomers, analogs, esters, amides, prodrugs, active metabolites, and derivatives thereof;

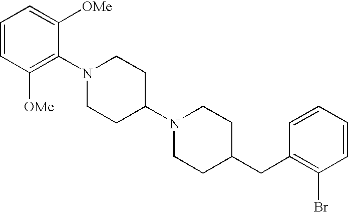

2. Thiophene and benzothiophene compounds (illustrated below) as disclosed in U.S. Pat. No. 6,262,056 and PCT Publication No. WO99/02516 and salts, enantiomers, analogs, esters, amides, prodrugs, active metabolites, and derivatives thereof;

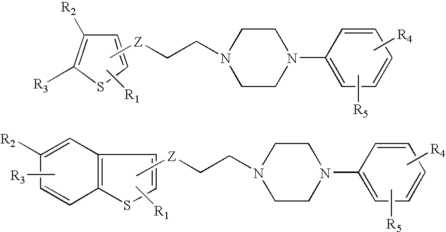

3. 3-[2-(1-(4′-piperonylpiperazinyl))indolyl]-carboxaldehydes (illustrated below) as disclosed in PCT Publication No. WO94/25454 and salts, enantiomers, analogs, esters, amides, prodrugs, active metabolites, and derivatives thereof;

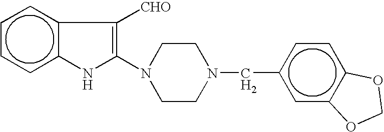

4. 3-[4-(3-substituted phenyl)piperazin-1-yl]-1-(benzo[b]thiophen-3-yl)propanol derivatives (illustrated below) as disclosed in Orus L et al. (2002) Pharmazie 57: 515-8 and salts, enantiomers, analogs, esters, amides, prodrugs, active metabolites, and derivatives thereof;

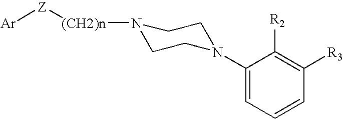

5. 1-aryl-3-[4-arylpiperazin-1-yl]-1-propane derivatives (illustrated below) as disclosed in Orus L et al. (2002) J Med Chem 45: 4128-39 and salts, enantiomers, analogs, esters, amides, prodrugs, active metabolites, and derivatives thereof, and

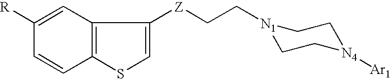

specifically including VN-53 and salts, enantiomers, analogs, esters, amides, prodrugs, active metabolites, and derivatives thereof (illustrated below);

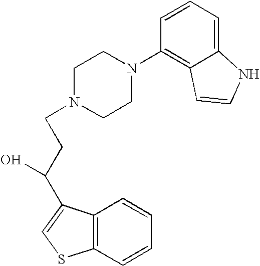

6. 3-[4-(aryl)piperazin-1-yl]-1-(benzo[b]thiophen-2-yl)propane derivatives (illustrated below) as disclosed in Orus L et al. (2002) Pharmazie 57: 355-7 and salts, enantiomers, analogs, esters, amides, prodrugs, active metabolites, and derivatives thereof;

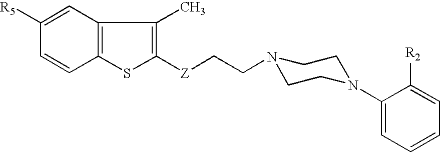

7. 1-aryl-3-(4-arylpiperazin-1-yl)propane derivatives (illustrated below) as disclosed in Martinez-Esparza J et al. (2001) J Med Chem 44: 418-28 and salts, enantiomers, analogs, esters, amides, prodrugs, active metabolites, and derivatives thereof;

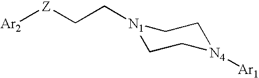

8. 3-[4-(aryl)piperazin-1-yl]-1-(benzo[b]thiophen-3-yl)propane derivatives (illustrated below) as disclosed in Martinez J et al. (2001) Eur J Med Chem 36: 55-61 and salts, enantiomers, analogs, esters, amides, prodrugs, active metabolites, and derivatives thereof;

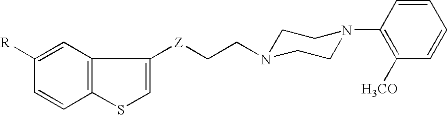

9. 3-[(4-aryl)piperazin-1-yl]-1-arylpropane derivatives (illustrated below) as disclosed in Oficialdegui A M et al. (2000) Farmaco 55: 345-53 and salts, enantiomers, analogs, esters, amides, prodrugs, active metabolites, and derivatives thereof;

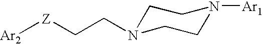

10. VN2222 (illustrated below) as disclosed in Tordera R M et al. (2002) Eur J Pharmacol 442: 63-71 and salts, enantiomers, analogs, esters, amides, prodrugs, active metabolites, and derivatives thereof;

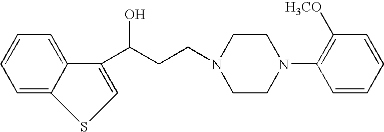

11. Arylpiperazinyl cyclohexyl derivatives (illustrated below) as disclosed in U.S. Pat. No. 6,465,482 and salts, enantiomers, analogs, esters, amides, prodrugs, active metabolites, and derivatives thereof;

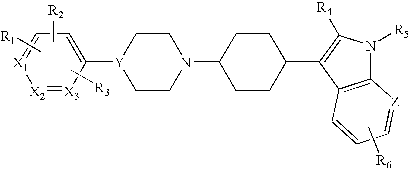

12. Aryloxy piperidinyl indole derivatives (illustrated below) as disclosed in U.S. Pat. No. 6,337,336 and salts, enantiomers, analogs, esters, amides, prodrugs, active metabolites, and derivatives thereof;

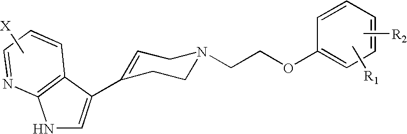

13. Arylpiperazinyl-cyclohexyl indole derivatives (illustrated below) as disclosed in U.S. Pat. No. 6,313,126 and salts, enantiomers, analogs, esters, amides, prodrugs, active metabolites, and derivatives thereof;

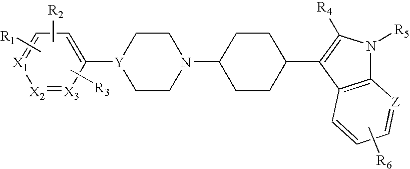

14. 3,4-Dihydro-2H-benzo[1,4]oxazinyl-methyl)-[3-(1H-indol-3yl)-alkyl]-amines (illustrated below) as disclosed in U.S. Pat. No. 6,313,114 and salts, enantiomers, analogs, esters, amides, prodrugs, active metabolites, and derivatives thereof;

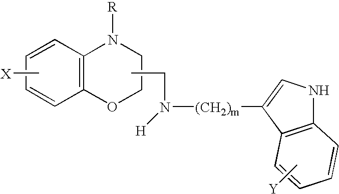

15. N-arloxyethyl-alkylamines (illustrated below) as disclosed in U.S. Pat. No. 6,291,683 and salts, enantiomers, analogs, esters, amides, prodrugs, active metabolites, and derivatives thereof;

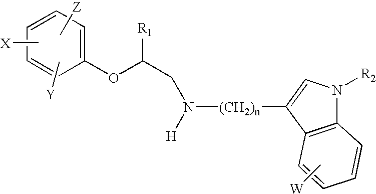

16. Tetrahydroisoquinolinyl-indole derivatives (illustrated below) as disclosed in U.S. Pat. No. 6,245,780 and salts, enantiomers, analogs, esters, amides, prodrugs, active metabolites, and derivatives thereof;

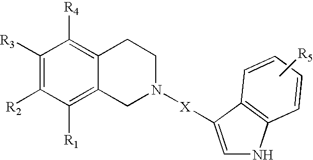

17. 3,4-Dihydro-2H-benzo[1,4]oxazine derivatives (illustrated below) as disclosed in U.S. Pat. No. 6,221,863 and salts, enantiomers, analogs, esters, amides, prodrugs, active metabolites, and derivatives thereof;

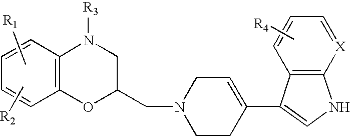

18. 1,4-disubstituted cyclohexane derivatives (illustrated below) as disclosed in U.S. Pat. No. 6,200,994 and salts, enantiomers, analogs, esters, amides, prodrugs, active metabolites, and derivatives thereof;

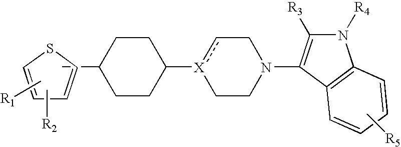

19. Indol-3-yl-cyclohexylamine derivatives (illustrated below) as disclosed in U.S. Pat. No. 6,162,803 and salts, enantiomers, analogs, esters, amides, prodrugs, active metabolites, and derivatives thereof;

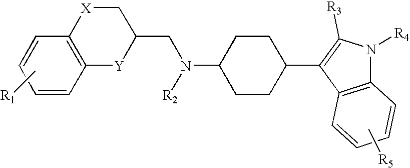

20. N-aryloxyethyl-indoly-alkylamines (illustrated below) as disclosed in U.S. Pat. No. 6,150,533 and salts, enantiomers, analogs, esters, amides, prodrugs, active metabolites, and derivatives thereof;

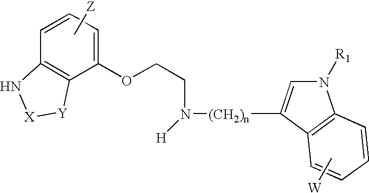

21. Aryloxyethyl-indoly-alkylamine derivatives (illustrated below) as disclosed in U.S. Pat. No. 6,121,307 and salts, enantiomers, analogs, esters, amides, prodrugs, active metabolites, and derivatives thereof;

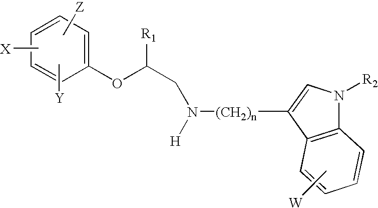

22. N-aryloxyethylarnine derivatives (illustrated below) as disclosed in U.S. Pat. No. 6,110,956 and salts, enantiomers, analogs, esters, amides, prodrugs, active metabolites, and derivatives thereof;

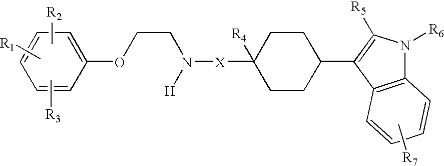

23. Aryl-8-azabicyclo[3.2.1]octanes (illustrated below) as disclosed in PCT Publication No. WO02/96906 and salts, enantiomers, analogs, esters, amides, prodrugs, active metabolites, and derivatives thereof;

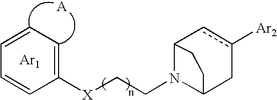

24. Azaindole derivatives (illustrated below) as disclosed in PCT Publication No. WO00/64898 and salts, enantiomers, analogs, esters, amides, prodrugs, active metabolites, and derivatives thereof;

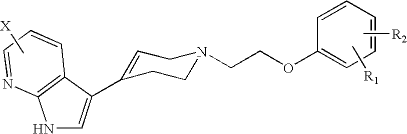

25. Dihydroisoquinolinyl-indole derivatives (illustrated below) as disclosed in PCT Publication No. WO00/64886 and salts, enantiomers, analogs, esters, amides, prodrugs, active metabolites, and derivatives thereof;

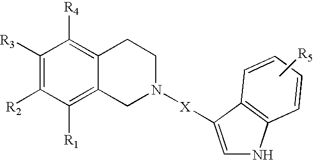

26. 3,4-dihydro-2H-benzo[1,4]oxazine derivatives (illustrated below) as disclosed in PCT Publication No. WO00/40581 and salts, enantiomers, analogs, esters, amides, prodrugs, active metabolites, and derivatives thereof;

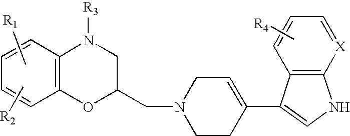

27. 3,4-dihydro-2Hbenzo[1,4]oxazinyl-methyl)-[3-(1H-indol-3-yl)-alkyl]amines (illustrated below) as disclosed in PCT Publication No. WO00/40580 and salts, enantiomers, analogs, esters, amides, prodrugs, active metabolites, and derivatives thereof;

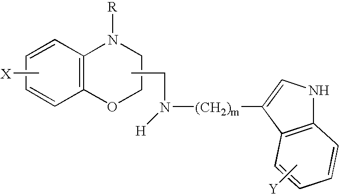

28. 1,4 disubstituted cyclohexane derivatives (illustrated below) as disclosed in PCT Publication No. WO00/40579 and salts, enantiomers, analogs, esters, amides, prodrugs, active metabolites, and derivatives thereof;

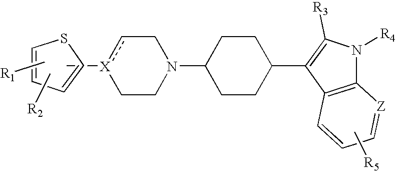

29. Arylpiperazinyl cyclohexyl derivatives (illustrated below) as disclosed in PCT Publication No. WO00/40554 and salts, enantiomers, analogs, esters, amides, prodrugs, active metabolites, and derivatives thereof;

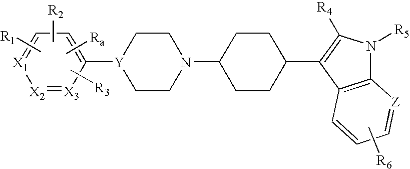

30. Indol-3-yl-cyclohexylamine derivatives (illustrated below) as disclosed in PCT Publication No. WO99/51592 and salts, enantiomers, analogs, esters, amides, prodrugs, active metabolites, and derivatives thereof;

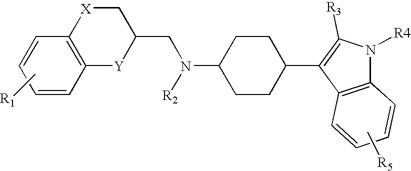

31. N-aryloxyethyl-indoly-alkylamines (illustrated below) as disclosed in PCT Publication No. WO99/51591 and salts, enantiomers, analogs, esters, amides, prodrugs, active metabolites, and derivatives thereof;

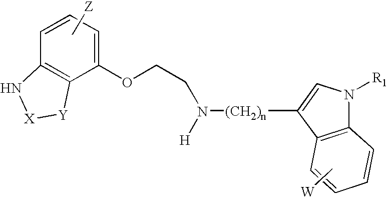

32. N-aryloxyethylamine derivatives (illustrated below) as disclosed in PCT Publication No. WO99/51576 and salts, enantiomers, analogs, esters, amides, prodrugs, active metabolites, and derivatives thereof;

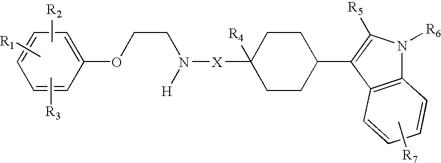

33. Aryloxyethyl-indoly-alkylamine derivatives (illustrated below) as disclosed in PCT Publication No. WO99/51575 and salts, enantiomers, analogs, esters, amides, prodrugs, active metabolites, and derivatives thereof;

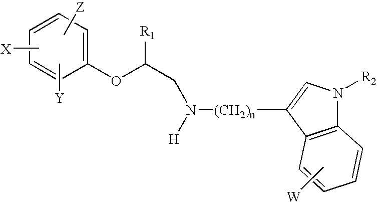

34. Substituted phenoxypropylamines (illustrated below) as disclosed in U.S. patent application No. 2002/0111358 and PCT Publication No. WO 02/42297 and salts, enantiomers, analogs, esters, amides, prodrugs, active metabolites, and derivatives thereof,

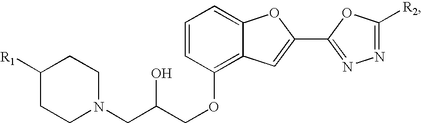

in particular, the two specific compounds shown below;

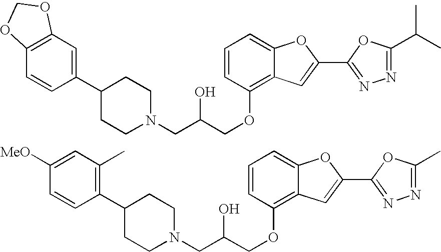

35. Substituted aminothienopyridines (illustrated below) as disclosed in U.S. Pat. No. 5,252,581 and salts, enantiomers, analogs, esters, amides, prodrugs, active metabolites, and derivatives thereof,

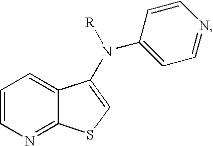

in particular, the two specific compounds shown below;

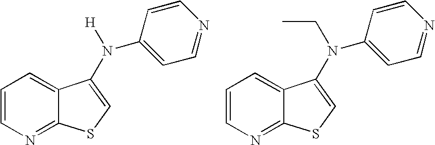

36. Aromatic amines of arylpiperazines (illustrated below) as disclosed in PCT Publication No. WO 98/23590 and salts, enantiomers, analogs, esters, amides, prodrugs, active metabolites, and derivatives thereof,

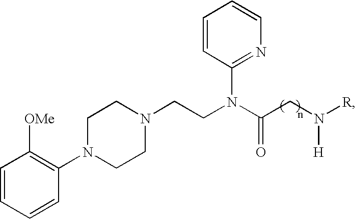

in particular, the two specific compounds shown below;

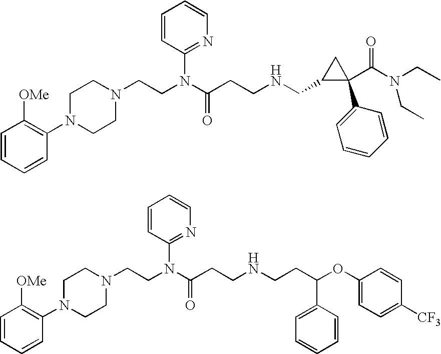

37. Piperidines and pyrrolidines (illustrated below) as disclosed in PCT Publication No. WO 97/40038 and salts, enantiomers, analogs, esters, amides, prodrugs, active metabolites, and derivatives thereof,

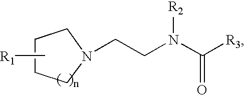

in particular, the compound shown below identified as (+)-MCU-629;

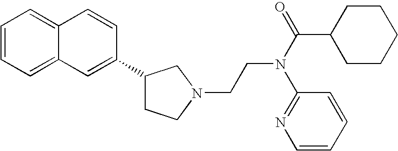

38. Benzoxazinone derivatives (illustrated below) as disclosed in PCT Publication No. WO 03/091248 and salts, enantiomers, analogs, esters, amides, prodrugs, active metabolites, and derivatives thereof;

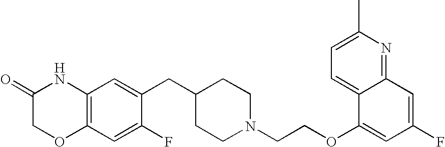

39. Indole derivatives (illustrated below) as disclosed in PCT Publication WO 01/46181 and salts, enantiomers, analogs, esters, amides, prodrugs, active metabolites, and derivatives thereof,

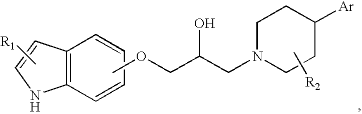

in particular, the specific compound shown below; and

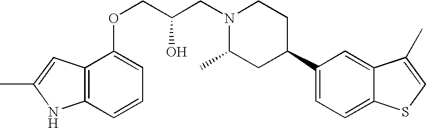

40. Tetrahydropyridine and piperazine derivatives (illustrated below) as disclosed in U.S. Pat. Nos. 6,596,722, 6,476,035, and 6,391,882, U.S. patent application Nos. 2002/0035113, 2002/0173512, and 2003/0018050, and PCT Publication Nos. WO 00/43382, WO 99/05140, and WO 99/67237 and salts, enantiomers, analogs, esters, amides, prodrugs, active metabolites, and derivatives thereof,

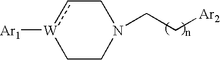

in particular, the specific compound shown below identified as LU-36-274.

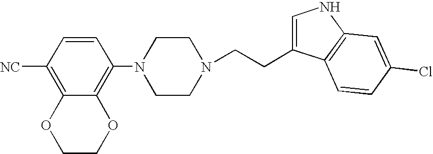

The identification of other agents useful in the present invention can be determined by methods known to one of skill in the art, including methods as disclosed in Lundmark J. et al. (1989) Acta Psychiatrica Scandinavica, Supplementum 350: 76-80, as well as those disclosed elsewhere herein.

Formulations

Formulations of the present invention are restricted to as-needed dosage forms, but may include short-term, rapid-onset, rapid-offset, controlled release, sustained release, delayed release, and pulsatile release formulations, so long as they are formulated to achieve as-needed administration of an active agent, as defined further herein. Formulations of the present invention may also include dosage forms for administering agents that are long-acting at an initial dose that achieves therapeutic plasma concentrations rapidly and the administration of that dose can be safely repeated as often as the patient desires, as defined elsewhere herein.

One or more additional active agents can be administered either simultaneously or sequentially with a compound exhibiting combined 5-HT_{1A} and SSRI activities, including rapid-onset compounds exhibiting combined 5-HT_{1A} receptor and SSRI activities and/or short acting compounds exhibiting combined 5-HT_{1A} receptor and SSRI activities. The additional active agent will generally, although not necessarily, be one that is effective in treating sexual dysfunction, particularly PE. The additional active agent could also be one that potentiates the effect of a compound exhibiting combined 5-HT_{1A} receptor and SSRI activities for treating sexual dysfunction, particularly PE. Suitable additional active agents include but are not limited to, for example, yohimbine, nitric oxide, eicosanoids (e.g., alprostadil), phosphodiesterase inhibitors (e.g., sildenafil citrate (VIAGRA®)), IC 351, and/or any agent that does not inhibit the action of the primary active agent.

Any of the active agents may be administered in the form of a salt, ester, amide, prodrug, active metabolite, derivative, or the like, provided that the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology, and involves reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are salts prepared with organic acids. Conversely, preparation of basic salts of acid moieties which may be present on an active agent are prepared in a similar manner using a pharmaceutically acceptable base such as sodium hydroxide, potassium hydroxide, ammonium hydroxide, calcium hydroxide, trimethylamine, or the like.

Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other salts, enantiomers, analogs, esters, amides, prodrugs, active metabolites, and derivatives of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Pharmaceutical Compositions and Dosage Forms

Suitable compositions and dosage forms include tablets, capsules, caplets, pills, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, rapidly disintegrating tablets including effervescent tablets or wafers, ointments, liquid formulations, foams and the like and the like. Further, those of ordinary skill in the art can readily deduce that suitable formulations involving these compositions and dosage forms, including those formulations as described elsewhere herein.

Oral Dosage Forms

Oral dosage forms include tablets, capsules, caplets, rapidly disintegrating tablets including effervescent tablets or wafers, solutions, suspensions and/or syrups, and may also comprise a plurality of granules, beads, powders or pellets that may or may not be encapsulated. Such dosage forms are prepared using conventional methods known to those in the field of pharmaceutical formulation and described in the pertinent texts, e.g., in Remington: The Science and Practice of Pharmacy, supra. Tablets and capsules represent the most convenient oral dosage forms, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), then, tablets prepared for oral administration using the method of the invention will generally contain other materials such as binders, diluents, lubricants, disintegrants, fillers, stabilizers, surfactants, preservatives, coloring agents, flavoring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts (See, for e.g., Remington: The Science and Practice of Pharmacy, supra). Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

The dosage form may also be a rapidly disintegrating tablet, including an effervescent tablet or wafer. Effervescent tablets are described in Remington, supra, and examples may be found in the literature, and in, for example, U.S. Pat. No. 5,211,957 to Hagemann et al. Generally, effervescent tablets contain the active agent in combination with additives such as sodium bicarbonate and an organic acid. e.g., tartaric acid or citric acid. In the presence of water, these additives react to liberate carbon dioxide thereby facilitating the disintegration of the tablet. Once the tablet is substantially disintegrated, an individual swallows the resultant solution thereby providing systemic adsorption of the active agent.

Another version of a rapidly disintegrating tablet includes “open matrix network” tablets. These tablets can disintegrate within seconds, i.e., within five to ten seconds, after being placed on the tongue of an individual. The contents of the tablet can then be swallowed with or without water. An example of such a tablet is found in U.S. Pat. No. 4,371,516 to Gregory et al. As described therein, the carrier provides a low density network, e.g., about 10 to about 200 mg/cm.sup.3, of water-soluble or water-dispersible material. The tablet is produced by subliming a solution containing both the drug and carrier that is subsequently directed to a mold having tablet-shaped depressions. The carrier may be any suitable material, but is preferably gelatin, with partially hydrolyzed gelatin most preferred. Other examples of rapidly disintegrating tablets that can be adapted to contain active agents as discloses herein are well-known in the art. See, for example, U.S. Pat. No. 5,776,492 to Betzing et al.

Transmucosal Compositions and Dosage Forms

Although the present compositions may be administered orally, other modes of administration are suitable as well. For example, transmucosal administration may be advantageously employed. Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible (hydrolyzable) polymeric carrier that may also serve to adhere the dosage form to the buccal mucosa. The buccal dosage unit is fabricated so as to erode over a predetermined time period, wherein drug delivery is provided essentially throughout. The time period for the present invention is typically in the range of from about 1 hour to about 12 hours. As-needed buccal drug delivery preferably will occur over a time period of from about 0 minutes to about 12 hours, more preferably from about 0 minutes to about 6 hours, and most preferably from about 0 minutes to about 4 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the active agent to be administered, and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other solid dosage form where the active ingredient is admixed into a disintegrable matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

For transurethral administration, the formulation comprises a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred.

Depending on the particular active agent administered, it may be desirable to incorporate a transurethral permeation enhancer in the urethral dosage form. Examples of suitable transurethral permeation enhancers include dimethylsulfoxide (“DMSO”), dimethyl formamide (“DMF”), N, N-dimethylacetamide (“DMA”), decylmethylsulfoxide (“C₁₀ MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone® from Nelson Research & Development Co., Irvine, Calif.), SEPA® (available from Macrochem Co., Lexington, Mass.), surfactants as discussed above, including, for example, Tergitol®, Nonoxynol-9® and TWEEN-80®, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug may also be contained in coatings, pellets or suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative may be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol. Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, it may also be preferred that urethral suppositories contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form comprises a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is on the order of from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. lontophoretically assisted drug delivery is, for example, described in PCT Publication No. WO 96/40054, cited above. Briefly, the active agent is driven through the urethral wall by means of an electric current passed from an external electrode to a second electrode contained within or affixed to a urethral probe.

Preferred transrectal dosage forms include rectal suppositories, creams, ointments, and liquid formulations (enemas). The suppository, cream, ointment or liquid formulation for transrectal delivery comprises a therapeutically effective amount of the selected phosphodiesterase inhibitor and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than about 3 hours.

Other components may also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

The active agents may also be administered intranasally or by inhalation. Compositions for intranasal administration are generally liquid formulations for administration as a spray or in the form of drops, although powder formulations for intranasal administration, e.g., insufflations, are also known, as are nasal gels, creams, pastes or ointments. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, simply by way of example, phosphate buffers. Furthermore, various devices are available in the art for the generation of drops, droplets and sprays, including droppers, squeeze bottles, and manually and electrically powered intranasal pump dispensers. Active agent containing intranasal carriers may also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, simply by way of example, alkylcelluloses and/or other biocompatible carriers of high viscosity well known to the art (see e.g., Remington: The Science and Practice of Pharmacy, supra). Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation may also comprise dry powder formulations, particularly insufflations in which the powder has an average particle size of from about 0.1 μm to about 50 μm, preferably from about 1 μm to about 25 μm.

Topical Formulations

Topical formulations may be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

Parenteral Administration

Parenteral administration, if used, is generally characterized by injection, including intramuscular, intraperitoneal, intravenous (IV) and subcutaneous injection. Injectable formulations can be prepared in conventional forms, either as liquid solutions or suspensions; solid forms suitable for solution or suspension in liquid prior to injection, or as emulsions. Preferably, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. A more recently revised approach for parenteral administration involves use of a slow release or sustained release system (See, e.g., U.S. Pat. No. 3,710,795).

Intravesical Administration

Intravesical administration, if used, is generally characterized by administration directly into the bladder and may include methods as described elsewhere herein. Other methods of intravesical administration may include those described in U.S. Pat. Nos. 6,207,180 and 6,039,967, as well as other methods that are known to one of skill in the art.

Intrathecal Administration

Intrathecal administration, if used, is generally characterized by administration directly into the intrathecal space (where fluid flows around the spinal cord).

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with sexual dysfunction, particularly PE.

Another system available from Medtronic that is commonly utilized for intrathecal administration is the is the fully implantable, programmable SynchroMed® Infusion System. The SynchroMed® Infuision System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It is made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed® pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription is stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration may include those described above available from Medtronic, as well as other methods that are known to one of skill in the art.

Additional Dosage Formulations and Drug Delivery Systems

As compared with traditional drug delivery approaches, some controlled release technologies rely upon the modification of both macromolecules and synthetic small molecules to allow them to be actively instead of passively absorbed into the body. For example, XenoPort Inc. utilizes technology that takes existing molecules and re-engineers them to create new chemical entities (unique molecules) that have improved pharmacologic properties to either: 1) lengthen the short half-life of a drug; 2) overcome poor absorption; and/or 3) deal with poor drug distribution to target tissues. Techniques to lengthen the short half-life of a drug include the use of prodrugs with slow cleavage rates to release drugs over time or that engage transporters in small and large intestines to allow the use of oral sustained delivery systems, as well as drugs that engage active transport systems. Examples of such controlled release formulations, tablets, dosage forms, and drug delivery systems, and that are suitable for use with the present invention, are described in the following published U.S. and PCT patent applications assigned to Xenoport Inc.: US20030158254; US20030158089; US20030017964; US2003130246; WO02100172; WO02100392; WO02100347; WO02100344; WO0242414; WO0228881; WO0228882; WO0244324; WO0232376; WO0228883; and WO0228411. Some other controlled release technologies rely upon methods that promote or enhance gastric retention, such as those developed by Depomed Inc. Because many drugs are best absorbed in the stomach and upper portions of the small intestine, Depomed has developed tablets that swell in the stomach during the postprandial or fed mode so that they are treated like undigested food. These tablets therefore sit safely and neutrally in the stomach for 6, 8, or more hours and deliver drug at a desired rate and time to upper gastrointestinal sites. Specific technologies in this area include: 1) tablets that slowly erode in gastric fluids to deliver drugs at almost a constant rate (particularly useful for highly insoluble drugs); 2) bi-layer tablets that combine drugs with different characteristics into a single table (such as a highly insoluble drug in an erosion layer and a soluble drug in a diffusion layer for sustained release of both); and 3) combination tablets that can either deliver drugs simultaneously or in sequence over a desired period of time (including an initial burst of a fast acting drug followed by slow and sustained delivery of another drug). Examples of such controlled release formulations that are suitable for use with the present invention and that rely upon gastric retention during the postprandial or fed mode, include tablets, dosage forms, and drug delivery systems in the following US patents assigned to Depomed Inc.: U.S. Pat. No. 6,488,962; U.S. Pat. No. 6,451,808; U.S. Pat. No. 6,340,475; U.S. Pat. No. 5,972,389; U.S. Pat. No. 5,582,837; and U.S. Pat. No. 5,007,790. Examples of such controlled release formulations that are suitable for use with the present invention and that rely upon gastric retention during the postprandial or fed mode, include tablets, dosage forms, and drug delivery systems in the following published U.S. and PCT patent applications assigned to Depomed Inc.: US20030147952; US20030104062; US20030104053; US20030104052; US20030091630; US20030044466; US20030039688; US20020051820; WO0335040; WO0335039; WO0156544; WO0132217; WO9855107; WO9747285; and WO9318755.

Other controlled release systems include those developed by ALZA Corporation based upon: 1) osmotic technology for oral delivery; 2) transdermal delivery via patches; 3) liposomal delivery via intravenous injection; 4) osmotic technology for long-term delivery via implants; and 5) depot technology designed to deliver agents for periods of days to a month. ALZA oral delivery systems include those that employ osmosis to provide precise, controlled drug delivery for up to 24 hours for both poorly soluble and highly soluble drugs, as well as those that deliver high drug doses meeting high drug loading requirements. ALZA controlled transdermal delivery systems provide drug delivery through intact skin for as long as one week with a single application to improve drug absorption and deliver constant amounts of drug into the bloodstream over time. ALZA liposomal delivery systems involve lipid nanoparticles that evade recognition by the immune system because of their unique polyethylene glycol (PEG) coating, allowing the precise delivery of drugs to disease-specific areas of the body. ALZA also has developed osmotically driven systems to enable the continuous delivery of small drugs, peptides, proteins, DNA and other bioactive macromolecules for up to one year for systemic or tissue-specific therapy. Finally, ALZA depot injection therapy is designed to deliver biopharmaceutical agents and small molecules for periods of days to a month using a nonaqueous polymer solution for the stabilization of macromolecules and a unique delivery profile.

Examples of controlled release formulations, tablets, dosage forms, and drug delivery systems that are suitable for use with the present invention are described in the following US patents assigned to ALZA Corporation: U.S. Pat. No. 4,367,741; U.S. Pat. No. 4,402,695; U.S. Pat. No. 4,418,038; U.S. Pat. No. 4,434,153; U.S. Pat. No. 4,439,199; U.S. Pat. No. 4,450,198; U.S. Pat. No. 4,455,142; U.S. Pat. No. 4,455,144; U.S. Pat. No. 4,484,923; U.S. Pat. No. 4,486,193; U.S. Pat. No. 4,489,197; U.S. Pat. No. 4,511,353; U.S. Pat. No. 4,519,801; U.S. Pat. No. 4,526,578; U.S. Pat. No. 4,526,933; U.S. Pat. No. 4,534,757; U.S. Pat. No. 4,553,973; U.S. Pat. No. 4,559,222; U.S. Pat. No. 4,564,364; U.S. Pat. No. 4,578,075; U.S. Pat. No. 4,588,580; U.S. Pat. No. 4,610,686; U.S. Pat. No. 4,618,487; U.S. Pat. No. 4,627,851; U.S. Pat. No. 4,629,449; U.S. Pat. No. 4,642,233; U.S. Pat. No. 4,649,043; U.S. Pat. No. 4,650,484; U.S. Pat. No. 4,659,558; U.S. Pat. No. 4,661,105; U.S. Pat. No. 4,662,880; U.S. Pat. No. 4,675,174; U.S. Pat. No. 4,681,583; U.S. Pat. No. 4,684,524; U.S. Pat. No. 4,692,336; U.S. Pat. No. 4,693,895; U.S. Pat. No. 4,704,119; U.S. Pat. No. 4,705,515; U.S. Pat. No. 4,717,566; U.S. Pat. No. 4,721,613; U.S. Pat. No. 4,723,957; U.S. Pat. No. 4,725,272; U.S. Pat. No. 4,728,498; U.S. Pat. No. 4,743,248; U.S. Pat. No. 4,747,847; U.S. Pat. No. 4,751,071; U.S. Pat. No. 4,753,802; U.S. Pat. No. 4,755,180; U.S. Pat. No. 4,756,314; U.S. Pat. No. 4,764,380; U.S. Pat. No. 4,773,907; U.S. Pat. No. 4,777,049; U.S. Pat. No. 4,781,924; U.S. Pat. No. 4,786,503; U.S. Pat. No. 4,788,062; U.S. Pat. No. 4,810,502; U.S. Pat. No. 4,812,313; U.S. Pat. No. 4,816,258; U.S. Pat. No. 4,824,675; U.S. Pat. No. 4,834,979; U.S. Pat. No. 4,837,027; U.S. Pat. No. 4,842,867; U.S. Pat. No. 4,846,826; U.S. Pat. No. 4,847,093; U.S. Pat. No. 4,849,226; U.S. Pat. No. 4,851,229; U.S. Pat. No. 4,851,231; U.S. Pat. No. 4,851,232; U.S. Pat. No. 4,853,229; U.S. Pat. No. 4,857,330; U.S. Pat. No. 4,859,470; U.S. Pat. No. 4,863,456; U.S. Pat. No. 4,863,744; U.S. Pat. No. 4,865,598; U.S. Pat. No. 4,867,969; U.S. Pat. No. 4,871,548; U.S. Pat. No. 4,872,873; U.S. Pat. No. 4,874,388; U.S. Pat. No. 4,876,093; U.S. Pat. No. 4,892,778; U.S. Pat. No. 4,902,514; U.S. Pat. No. 4,904,474; U.S. Pat. No. 4,913,903; U.S. Pat. No. 4,915,949; U.S. Pat. No. 4,915,952; U.S. Pat. No. 4,917,895; U.S. Pat. No. 4,931,285; U.S. Pat. No. 4,946,685; U.S. Pat. No. 4,948,592; U.S. Pat. No. 4,954,344; U.S. Pat. No. 4,957,494; U.S. Pat. No. 4,960,416; U.S. Pat. No. 4,961,931; U.S. Pat. No. 4,961,932; U.S. Pat. No. 4,963,141; U.S. Pat. No. 4,966,769; U.S. Pat. No. 4,971,790; U.S. Pat. No. 4,976,966; U.S. Pat. No. 4,986,987; U.S. Pat. No. 5,006,346; U.S. Pat. No. 5,017,381; U.S. Pat. No. 5,019,397; U.S. Pat. No. 5,023,076; U.S. Pat. No. 5,023,088; U.S. Pat. No. 5,024,842; U.S. Pat. No. 5,028,434; U.S. Pat. No. 5,030,454; U.S. Pat. No. 5,071,656; U.S. Pat. No. 5,077,054; U.S. Pat. No. 5,082,668; U.S. Pat. No. 5,104,390; U.S. Pat. No. 5,110,597; U.S. Pat. No. 5,122,128; U.S. Pat. No. 5,125,894; U.S. Pat. No. 5,141,750; U.S. Pat. No. 5,141,752; U.S. Pat. No. 5,156,850; U.S. Pat. No. 5,160,743; U.S. Pat. No. 5,160,744; U.S. Pat. No. 5,169,382; U.S. Pat. No. 5,171,576; U.S. Pat. No. 5,176,665; U.S. Pat. No. 5,185,158; U.S. Pat. No. 5,190,765; U.S. Pat. No. 5,198,223; U.S. Pat. No. 5,198,229; U.S. Pat. No. 5,200,195; U.S. Pat. No. 5,200,196; U.S. Pat. No. 5,204,116; U.S. Pat. No. 5,208,037; U.S. Pat. No. 5,209,746; U.S. Pat. No. 5,221,254; U.S. Pat. No. 5,221,278; U.S. Pat. No. 5,229,133; U.S. Pat. No. 5,232,438; U.S. Pat. No. 5,232,705; U.S. Pat. No. 5,236,689; U.S. Pat. No. 5,236,714; U.S. Pat. No. 5,240,713; U.S. Pat. No. 5,246,710; U.S. Pat. No. 5,246,711; U.S. Pat. No. 5,252,338; U.S. Pat. No. 5,254,349; U.S. Pat. No. 5,266,332; U.S. Pat. No. 5,273,752; U.S. Pat. No. 5,284,660; U.S. Pat. No. 5,286,491; U.S. Pat. No. 5,308,348; U.S. Pat. No. 5,318,558; U.S. Pat. No. 5,320,850; U.S. Pat. No. 5,322,502; U.S. Pat. No. 5,326,571; U.S. Pat. No. 5,330,762; U.S. Pat. No. 5,338,550; U.S. Pat. No. 5,340,590; U.S. Pat. No. 5,342,623; U.S. Pat. No. 5,344,656; U.S. Pat. No. 5,348,746; U.S. Pat. No. 5,358,721; U.S. Pat. No. 5,364,630; U.S. Pat. No. 5,376,377; U.S. Pat. No. 5,391,381; U.S. Pat. No. 5,402,777; U.S. Pat. No. 5,403,275; U.S. Pat. No. 5,411,740; U.S. Pat. No. 5,417,675; U.S. Pat. No. 5,417,676; U.S. Pat. No. 5,417,682; U.S. Pat. No. 5,423,739; U.S. Pat. No. 5,424,289; U.S. Pat. No. 5,431,919; U.S. Pat. No. 5,443,442; U.S. Pat. No. 5,443,459; U.S. Pat. No. 5,443,461; U.S. Pat. No. 5,456,679; U.S. Pat. No. 5,460,826; U.S. Pat. No. 5,462,741; U.S. Pat. No. 5,462,745; U.S. Pat. No. 5,489,281; U.S. Pat. No. 5,499,979; U.S. Pat. No. 5,500,222; U.S. Pat. No. 5,512,293; U.S. Pat. No. 5,512,299; U.S. Pat. No. 5,529,787; U.S. Pat. No. 5,531,736; U.S. Pat. No. 5,532,003; U.S. Pat. No. 5,533,971; U.S. Pat. No. 5,534,263; U.S. Pat. No. 5,540,912; U.S. Pat. No. 5,543,156; U.S. Pat. No. 5,571,525; U.S. Pat. No. 5,573,503; U.S. Pat. No. 5,591,124; U.S. Pat. No. 5,593,695; U.S. Pat. No. 5,595,759; U.S. Pat. No. 5,603,954; U.S. Pat. No. 5,607,696; U.S. Pat. No. 5,609,885; U.S. Pat. No. 5,614,211; U.S. Pat. No. 5,614,578; U.S. Pat. No. 5,620,705; U.S. Pat. No. 5,620,708; U.S. Pat. No. 5,622,530; U.S. Pat. No. 5,622,944; U.S. Pat. No. 5,633,011; U.S. Pat. No. 5,639,477; U.S. Pat. No. 5,660,861; U.S. Pat. No. 5,667,804; U.S. Pat. No. 5,667,805; U.S. Pat. No. 5,674,895; U.S. Pat. No. 5,688,518; U.S. Pat. No. 5,698,224; U.S. Pat. No. 5,702,725; U.S. Pat. No. 5,702,727; U.S. Pat. No. 5,707,663; U.S. Pat. No. 5,713,852; U.S. Pat. No. 5,718,700; U.S. Pat. No. 5,736,580; U.S. Pat. No. 5,770,227; U.S. Pat. No. 5,780,058; U.S. Pat. No. 5,783,213; U.S. Pat. No. 5,785,994; U.S. Pat. No. 5,795,591; U.S. Pat. No. 5,811,465; U.S. Pat. No. 5,817,624; U.S. Pat. No. 5,824,340; U.S. Pat. No. 5,830,501; U.S. Pat. No. 5,830,502; U.S. Pat. No. 5,840,754; U.S. Pat. No. 5,858,407; U.S. Pat. No. 5,861,439; U.S. Pat. No. 5,863,558; U.S. Pat. No. 5,876,750; U.S. Pat. No. 5,883,135; U.S. Pat. No. 5,897,878; U.S. Pat. No. 5,904,934; U.S. Pat. No. 5,904,935; U.S. Pat. No. 5,906,832; U.S. Pat. No. 5,912,268; U.S. Pat. No. 5,914,131; U.S. Pat. No. 5,916,582; U.S. Pat. No. 5,932,547; U.S. Pat. No. 5,938,654; U.S. Pat. No. 5,941,844; U.S. Pat. No. 5,955,103; U.S. Pat. No. 5,972,369; U.S. Pat. No. 5,972,370; U.S. Pat. No. 5,972,379; U.S. Pat. No. 5,980,943; U.S. Pat. No. 5,981,489; U.S. Pat. No. 5,983,130; U.S. Pat. No. 5,989,590; U.S. Pat. No. 5,995,869; U.S. Pat. No. 5,997,902; U.S. Pat. No. 6,001,390; U.S. Pat. No. 6,004,309; U.S. Pat. No. 6,004,578; U.S. Pat. No. 6,008,187; U.S. Pat. No. 6,020,000; U.S. Pat. No. 6,034,101; U.S. Pat. No. 6,036,973; U.S. Pat. No. 6,039,977; U.S. Pat. No. 6,057,374; U.S. Pat. No. 6,066,619; U.S. Pat. No. 6,068,850; U.S. Pat. No. 6,077,538; U.S. Pat. No. 6,083,190; U.S. Pat. No. 6,096,339; U.S. Pat. No. 6,106,845; U.S. Pat. No. 6,110,499; U.S. Pat. No. 6,120,798; U.S. Pat. No. 6,120,803; U.S. Pat. No. 6,124,261; U.S. Pat. No. 6,130,200; U.S. Pat. No. 6,146,662; U.S. Pat. No. 6,153,678; U.S. Pat. No. 6,174,547; U.S. Pat. No. 6,183,466; U.S. Pat. No. 6,203,817; U.S. Pat. No. 6,210,712; U.S. Pat. No. 6,210,713; U.S. Pat. No. 6,224,907; U.S. Pat. No. 6,235,712; U.S. Pat. No. 6,245,357; U.S. Pat. No. 6,262,115; U.S. Pat. No. 6,264,990; U.S. Pat. No. 6,267,984; U.S. Pat. No. 6,287,598; U.S. Pat. No. 6,289,241; U.S. Pat. No. 6,331,311; U.S. Pat. No. 6,333,050; U.S. Pat. No. 6,342,249; U.S. Pat. No. 6,346,270; U.S. Pat. No. 6365183; U.S. Pat. No. 6,368,626; U.S. Pat. No. 6,387,403; U.S. Pat. No. 6,419,952; U.S. Pat. No. 6,440,457; U.S. Pat. No. 6,468,961; U.S. Pat. No. 6,491,683; U.S. Pat. No. 6,512,010; U.S. Pat. No. 6,514,530; U.S. Pat. No. 6,534,089; U.S. Pat. No. 6,544,252; U.S. Pat. No. 6,548,083; U.S. Pat. No. 6,551,613; U.S. Pat. No. 6,572,879; and U.S. Pat. No. 6,596,314.

Other examples of controlled release formulations, tablets, dosage forms, and drug delivery systems that are suitable for use with the present invention are described in the following published U.S. patent application and PCT applications assigned to ALZA Corporation: US20010051183; WO0004886; WO0013663; WO0013674; WO0025753; WO0025790; WO0035419; WO0038650; WO0040218; WO0045790; WO0066126; WO0074650; WO0119337; WO0119352; WO0121211; WO0137815; WO0141742; WO0143721; WO0156543; WO3041684; WO03041685; WO03041757; WO03045352; WO03051341; WO03053400; WO03053401; WO9000416; WO9004965; WO9113613; WO9116884; WO9204011; WO9211843; WO9212692; WO9213521; WO9217239; WO9218102; WO9300071; WO9305843; WO9306819; WO9314813; WO9319739; WO9320127; WO9320134; WO9407562; WO9408572; WO9416699; WO9421262; WO9427587; WO9427589; WO9503823; WO9519174; WO9529665; WO9600065; WO9613248; WO9625922; WO9637202; WO9640049; WO9640050; WO9640139; WO9640364; WO9640365; WO9703634; WO9800158; WO9802169; WO9814168; WO9816250; WO9817315; WO9827962; WO9827963; WO9843611; WO9907342; WO9912526; WO9912527; WO9918159; WO9929297; WO9929348; WO9932096; WO9932153; WO9948494; WO9956730; WO9958115; and WO9962496.

Andrx Corporation has also developed drug delivery technology suitable for use in the present invention that includes: 1) a pelletized pulsatile delivery system (“PPDS”); 2) a single composition osmotic tablet system (“SCOT”); 3) a solubility modulating hydrogel system (“SMHS”); 4) a delayed pulsatile hydrogel system (“DPHS”); 5) a stabilized pellet delivery system (“SPDS”); 6) a granulated modulating hydrogel system (“GMHS”); 7) a pelletized tablet system (“PELTAB”); 8) a porous tablet system (“PORTAB”); and 9) a stabilized tablet delivery system (“STDS”). PPDS uses pellets that are coated with specific polymers and agents to control the release rate of the microencapsulated drug and is designed for use with drugs that require a pulsed release. SCOT utilizes various osmotic modulating agents as well as polymer coatings to provide a zero-order drug release. SMHS utilizes a hydrogel-based dosage system that avoids the “initial burst effect” commonly observed with other sustained-release hydrogel formulations and that provides for sustained release without the need to use special coatings or structures that add to the cost of manufacturing. DPHS is designed for use with hydrogel matrix products characterized by an initial zero-order drug release followed by a rapid release that is achieved by the blending of selected hydrogel polymers to achieve a delayed pulse. SPDS incorporates a pellet core of drug and protective polymer outer layer, and is designed specifically for unstable drugs, while GMHS incorporates hydrogel and binding polymers with the drug and forms granules that are pressed into tablet form. PELTAB provides controlled release by using a water insoluble polymer to coat discrete drug crystals or pellets to enable them to resist the action of fluids in the gastrointestinal tract, and these coated pellets are then compressed into tablets. PORTAB provides controlled release by incorporating an osmotic core with a continuous polymer coating and a water soluble component that expands the core and creates microporous channels through which drug is released. Finally, STDS includes a dual layer coating technique that avoids the need to use a coating layer to separate the enteric coating layer from the omeprazole core.

Examples of controlled release formulations, tablets, dosage forms, and drug delivery systems that are suitable for use with the present invention are described in the following US patents assigned to Andrx Corporation: U.S. Pat. No. 5,397,574; U.S. Pat. No. 5,419,917; U.S. Pat. No. 5,458,887; U.S. Pat. No. 5,458,888; U.S. Pat. No. 5,472,708; U.S. Pat. No. 5,508,040; U.S. Pat. No. 5,558,879; U.S. Pat. No. 5,567,441; U.S. Pat. No. 5,654,005; U.S. Pat. No. 5,728,402; U.S. Pat. No. 5,736,159; U.S. Pat. No. 5,830,503; U.S. Pat. No. 5,834,023; U.S. Pat. No. 5,837,379; U.S. Pat. No. 5,916,595; U.S. Pat. No. 5,922,352; U.S. Pat. No. 6,099,859; U.S. Pat. No. 6,099,862; U.S. Pat. No. 6,103,263; U.S. Pat. No. 6,106,862; U.S. Pat. No. 6,156,342; U.S. Pat. No. 6,177,102; U.S. Pat. No. 6,197,347; U.S. Pat. No. 6,210,716; U.S. Pat. No. 6,238,703; U.S. Pat. No. 6,270,805; U.S. Pat. No. 6,284,275; U.S. Pat. No. 6,485,748; U.S. Pat. No. 6,495,162; U.S. Pat. No. 6,524,620; U.S. Pat. No. 6,544,556; U.S. Pat. No. 6,589,553; U.S. Pat. No. 6,602,522; and U.S. Pat. No. 6,610,326.

Examples of controlled release formulations, tablets, dosage forms, and drug delivery systems that are suitable for use with the present invention are described in the following published US and PCT patent applications assigned to Andrx Corporation: US20010024659; US 20020115718; US 20020156066; WO0004883; WO0009091; WO0012097; WO0027370; WO0050010; WO0132161; WO0134123; WO0236077; WO0236100; WO02062299; WO02062824; WO02065991; WO02069888; WO02074285; WO03000177; WO9521607; WO9629992; WO9633700; WO9640080; WO9748386; WO9833488; WO9833489; WO9930692; WO9947125; and WO9961005.

Some other examples of drug delivery approaches focus on non-oral drug delivery, providing parenteral, transmucosal, and topical delivery of proteins, peptides, and small molecules. For example, the Atrigel® drug delivery system marketed by Atrix Laboratories Inc. comprises biodegradable polymers, similar to those used in biodegradable sutures, dissolved in biocompatible carriers. These pharmaceuticals may be blended into a liquid delivery system at the time of manufacturing or, depending upon the product, may be added later by a physician at the time of use. Injection of the liquid product subcutaneously or intramuscularly through a small gauge needle, or placement into accessible tissue sites through a cannula, causes displacement of the carrier with water in the tissue fluids, and a subsequent precipitate to form from the polymer into a solid film or implant. The drug encapsulated within the implant is then released in a controlled manner as the polymer matrix biodegrades over a period ranging from days to months. Examples of such drug delivery systems include Atrix's Eligard®, Atridox®/Doxirobe®, Atrisorb® FreeFlow™/Atrisorb®-D FreeFlow, bone growth products, and others as described in the following published US and PCT patent applications assigned to Atrix Laboratories Inc.: US RE37950; U.S. Pat. No. 6,630,155; U.S. Pat. No. 6,566,144; U.S. Pat. No. 6,610,252; U.S. Pat. No. 6,565,874; U.S. Pat. No. 6,528,080; U.S. Pat. No. 6,461,631; U.S. Pat. No. 6,395,293; U.S. Pat. No. 6,261,583; U.S. Pat. No. 6,143,314; U.S. Pat. No. 6,120,789; U.S. Pat. No. 6,071,530; U.S. Pat. No. 5,990,194; U.S. Pat. No. 5,945,115; U.S. Pat. No. 5,888,533; U.S. Pat. No. 5,792,469; U.S. Pat. No. 5,780,044; U.S. Pat. No. 5,759,563; U.S. Pat. No. 5,744,153; U.S. Pat. No. 5,739,176; U.S. Pat. No. 5,736,152; U.S. Pat. No. 5,733,950; U.S. Pat. No. 5,702,716; U.S. Pat. No. 5,681,873; U.S. Pat. No. 5,660,849; U.S. Pat. No. 5,599,552; U.S. Pat. No. 5,487,897; U.S. Pat. No. 5,368,859; U.S. Pat. No. 5,340,849; U.S. Pat. No. 5,324,519; U.S. Pat. No. 5,278,202; U.S. Pat. No. 5,278,201; US 20020114737, US 20030195489; US 20030133964; US20010042317; US 20020090398; US20020001608; and US2001042317.

Atrix Laboratories Inc. also markets technology for the non-oral transmucosal delivery of drugs over a time period from minutes to hours. For example, Atrix's BEMA™ (Bioerodible Muco-Adhesive Disc) drug delivery system comprises pre-formed bioerodible discs for local or systemic delivery. Examples of such drug delivery systems include those as described in U.S. Pat. No. 6,245,345.

Other drug delivery systems marketed by Atrix Laboratories Inc. focus on topical drug delivery. For example, SMPT™ (Solvent Particle System) allows the topical delivery of highly water-insoluble drugs. This product allows for a controlled amount of a dissolved drug to permeate the epidermal layer of the skin by combining the dissolved drug with a microparticle suspension of the drug. The SMP™ system works in stages whereby: 1) the product is applied to the skin surface; 2) the product near follicles concentrates at the skin pore; 3) the drug readily partitions into skin oils; and 4) the drug diffuses throughout the area. By contrast, MCA® (Mucocutaneous Absorption System) is a water-resistant topical gel providing sustained drug delivery. MCA® forms a tenacious film for either wet or dry surfaces where: 1) the product is applied to the skin or mucosal surface; 2) the product forms a tenacious moisture-resistant film; and 3) the adhered film provides sustained release of drug for a period from hours to days. Yet another product, BCP™ (Biocompatible Polymer System) provides a non-cytotoxic gel or liquid that is applied as a protective film for wound healing. Examples of these systems include Orajel®-Ultra Mouth Sore Medicine as well as those as described in the following published U.S. patents and applications assigned to Atrix Laboratories Inc.: U.S. Pat. No. 6,537,565; U.S. Pat. No. 6,432,415; U.S. Pat. No. 6,355,657; U.S. Pat. No. 5,962,006; U.S. Pat. No. 5,725,491; U.S. Pat. No. 5,722,950; U.S. Pat. No. 5,717,030; U.S. Pat. No. 5,707,647; U.S. Pat. No. 5,632,727; and US 20010033853.

Dosage and Administration

The concentration of the active agent in any of the aforementioned dosage forms and compositions can vary a great deal, and will depend on a variety of factors, including the type of composition or dosage form, the corresponding mode of administration, the nature and activity of the specific active agent, and the intended drug release profile. Preferred dosage forms contain a unit dose of active agent, i.e., a single therapeutically effective dose. For creams, ointments, etc., a “unit dose” requires an active agent concentration that provides a unit dose in a specified quantity of the formulation to be applied. The unit dose of any particular active agent will depend, of course, on the active agent and on the mode of administration.

For compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for oral administration will be in the range of from about 1 mg to about 10,000 mg, typically in the range of from about 100 mg to about 5,000 mg; for local administration, suitable unit doses may be lower. Alternatively, for compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for oral administration will be greater than about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. Those of ordinary skill in the art of pharmaceutical formulation can readily deduce suitable unit doses for other compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires. One of ordinary skill in the art of pharmaceutical formulation can also readily deduce suitable unit doses for other types of active agents that may be incorporated into a dosage form of the invention.

For compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for transmucosal, topical, transdermal, and parenteral administration will be in the range of from about 1 ng to about 10,000 mg, typically in the range of from about 100 ng to about 5,000 mg. Alternatively, for compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for transmucosal, topical, transdermal, and parenteral administration will be greater than about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, about 500 μg, about 1 mg, about 5 mg, about 10 mg, about 20 mg, about 30 mg, about 40 mg, about 50 mg, about 100 mg, about 200 mg, about 300 mg, about 400 mg, about 500 mg, about 1,000 mg, about 1,500 mg, about 2,000 mg, about 2,500 mg, about 3,000 mg, about 3,500 mg, about 4,000 mg, about 4,500 mg, about 5,000 mg, about 5,500 mg, about 6,000 mg, about 6,500 mg, about 7,000 mg, about 7,500 mg, about 8,000 mg, about 8,500 mg, about 9,000 mg, or about 9,500 mg. Those of ordinary skill in the art of pharmaceutical formulation can readily deduce suitable unit doses for other compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires. One of ordinary skill in the art of pharmaceutical formulation can also readily deduce suitable unit doses for other types of active agents that may be incorporated into a dosage form of the invention.

For compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for intrathecal administration will be in the range of from about 1 fg to about 1 mg, typically in the range of from about 100 fg to about 1 ng. Alternatively, for compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, the unit dose for intrathecal administration will be greater than about 1 fg, about 5 fg, about 10 fg, about 20 fg, about 30 fg, about 40 fg, about 50 fg, about 100 fg, about 200 fg, about 300 fg, about 400 fg, about 500 fg, about 1 pg, about 5 pg, about 10 pg, about 20 pg, about 30 pg, about 40 pg, about 50 pg, about 100 pg, about 200 pg, about 300 pg, about 400 pg, about 500 pg, about 1 ng, about 5 ng, about 10 ng, about 20 ng, about 30 ng, about 40 ng, about 50 ng, about 100 ng, about 200 ng, about 300 ng, about 400 ng, about 500 ng, about 1 μg, about 5 μg, about 10 μg, about 20 μg, about 30 μg, about 40 μg, about 50 μg, about 100 μg, about 200 μg, about 300 μg, about 400 μg, or about 500 μg. Those of ordinary skill in the art of pharmaceutical formulation can readily deduce suitable unit doses for other compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires. One of ordinary skill in the art of pharmaceutical formulation can also readily deduce suitable unit doses for other types of active agents that may be incorporated into a dosage form of the invention.

A therapeutically effective amount of a particular active agent administered to a given individual will, of course, be dependent on a number of factors, including the concentration of the specific active agent, composition or dosage form, the selected mode of administration, the age and general condition of the individual being treated, the severity of the individual's condition, and other factors known to the prescribing physician. However, one of skill in the art would readily recognize that the therapeutically effective amount of a particular active agent must be selected so as to allow for as-needed administration, as defined further herein.

With an immediate release dosage form, as-needed administration may involve drug administration immediately prior to commencement of an activity wherein suppression of the symptoms of sexual dysfunction, particularly PE, would be desirable, but will generally be in the range of from about 0 minutes to about 10 hours prior to such an activity, preferably in the range of from about 0 minutes to about 6 hours prior to such an activity, most preferably in the range of from about 0 minutes to about 4 hours prior to such an activity.

Packaged Kits

In another embodiment, a packaged kit is provided that contains the pharmaceutical formulation to be administered, i.e., a pharmaceutical formulation containing a therapeutically effective amount of a selected active agent for the treatment of sexual dysfumction, particularly PE, a container, preferably sealed, for housing the formulation during storage and prior to use, and instructions for carrying out drug administration in a manner effective to treat sexual dysfunction, particularly PE. The instructions will typically be written instructions on a package insert and/or on a label. Depending on the type of formulation and the intended mode of administration, the kit may also include a device for administering the formulation. The formulation may be any suitable formulation as described herein. For example, the formulation may be an oral dosage form containing a unit dosage of a selected active agent. The kit may contain multiple formulations of different dosages of the same agent. The kit may also contain multiple formulations of different active agents.

Insurance Claims

In general, the processing of an insurance claim for the coverage of a given medical treatment or drug therapy involves notification of the insurance company, or any other entity, that has issued the insurance policy against which the claim is being filed, that the medical treatment or drug therapy will be performed. A determination is then made as to whether the medical treatment or drug therapy that will be performed is covered under the terms of the policy. If covered, the claim is then processed, which can include payment, reimbursement, or application against a deductable.

The present invention encompasses a method for processing an insurance claim under an insurance policy for compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, or pharmaceutically acceptable salts, esters, amides, prodrugs, or active metabolites thereof used in the treatment of sexual dysfunction, particularly PE. This method comprises: 1) receiving notification that treatment of sexual dysfunction, particularly PE, using said compounds exhibiting combined 5-HT_{1A} receptor and SSRI activities, or pharmaceutically acceptable salts, esters, amides, prodrugs or active metabolites thereof will be performed or receiving notification of a prescription for said compounds exhibiting combined 5-HT_{1A} receptor and SSRI activities to treat sexual dysfunction, particularly PE; 2) determining whether said treatment using said compounds exhibiting combined 5-HT_{1A} receptor and SSRI activities, or pharmaceutically acceptable salts, esters, amides, prodrugs or active metabolites is covered under said insurance policy; and 3) processing said claim for treatment using said compounds exhibiting combined 5-HT_{1A} receptor and SSRI activities or pharmaceutically acceptable salts, esters, amides, prodrugs, or active metabolites thereof, including payment, reimbursement, or application against a deductable.

The present invention also encompasses the method for processing an insurance claim described above, wherein compounds exhibiting combined 5-HT_{1A} receptor and SSRI activities and a secondary agent are used in the treatment of sexual dysfunction, particularly PE. Secondary agents can include yohimbine, nitric oxide, eicosanoids (e.g., alprostadil), phosphodiesterase inhibitors (e.g., sildenafil citrate (VIAGRA®)), IC 351, any agent that does not inhibit the action of the primary active agent, or pharmaceutically acceptable salts, esters, amides, prodrugs, or active metabolites thereof. Futhermore, the method for processing an insurance claim according to the present invention encompasses wherein said compounds exhibiting combined 5-HT_{1A} receptor and SSRI activities and said secondary agent, or pharmaceutically acceptable salts, esters, amides, prodrugs, or active metabolites thereof, are administered sequentially, concurrently in the same composition, or concurrently in different compositions. The method for processing an insurance claim according to the present invention also encompasses the processing of claims for compounds exhibiting combined 5-HT_{1A} receptor and SSRI activities, particularly compounds exhibiting combined 5-HT_{1A} receptor and SSRI activities and one of the secondary agents described above, or pharmaceutically acceptable salts, esters, amides, prodrugs, or active metabolites thereof, when either has been prescribed separately or concurrently for the treatment of sexual dysfunction, particularly PE.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

All patents, patent applications, and publications mentioned herein are hereby incorporated by reference in their entireties.

### EXAMPLE 1

### Behavioral Model of Sexual Dysfunction

Objective and Rationale

The objective of the current study is to determine the effect of compounds exhibiting combined 5-HT_{1A} receptor and SSRI activities, including rapid-onset compounds exhibiting combined 5-HT_{1A} receptor and SSRI activities and/or short acting compounds exhibiting combined 5-HT_{1A} receptor and SSRI activities, as well as agents that are long-acting but can be administered at an initial dose that achieves therapeutic plasma concentrations rapidly and the administration of that dose can be safely repeated as often as the patient desires, to treat sexual dysfunction, particularly PE.

These methods include the use of a well accepted model for sexual dysfumction, including PE, involving monitoring the motivation, proceptivity and consummatory behavior of male rats as described in Ahlemius & Larsson (1999) Europ. J. Pharmacol. 379: 1-6. The model encompasses partnership of a male with a female and examination of their respective behaviors. The females are ovariectomized and hormonally primed in order to make them receptive, thus allowing consummatory sexual behavior to take place. The male is treated with the compounds or vehicle and then sexual behavior monitored.

Methods

All rats are housed in approved animal facilities at UNC Chapel Hill. All rats are acclimatized to a reversed light cycle. Female rats are ovariectomized 7 days prior to behavioral testing. Females are primed with estrogen and progesterone 48 and 2 hours, respectively, prior to contact with the males. Behavioral testing takes place in the early dark period. Male Sprague Dawley rats are tested initially to verify that they reproducibly ejaculate (3 times over a 2-3 week period) upon placement with a receptive female. Males that do not consistently show ejaculatory responses are not included in these studies.

Males are treated with an active agent or vehicle (1-60 min before testing). Males are placed into the testing chamber 5 min before introducing the female. Females are placed with the male and behavior is recorded for 45-60 min. (depending on the half-life of the compound) with a camcorder. If a male does not mount the female or the female does not respond to the male with lordosis, within 5 min. then the female is replaced. Up to 3 females are tried with each male. Rats are retested around 7 days after their previous behavioral recordings.

Video tapes are subsequently examined and the number of attempted mounts, mounts, intromissions and ejaculations are recorded. The latency to the first intromission and each ejaculation is calculated. The post-ejaculatory interval is calculated. Data is compared using t-test and one-way ANOVA as appropriate.

There are approximately 6 groups of animals, with each group designated a specific drug. Four drug doses are tested. One to two trials with the vehicle control are given to each animal. Animals receive the compounds and vehicle in random order. The time required for this study includes acclimatization time for the animals after arrival (2 weeks), ovariectomy (2 weeks), testing the males to give them practice with females (3 weeks), study duration (approximately 6 weeks), and data analysis (approximately 15 weeks). The total time required for the study is therefore approximately 28 weeks.

What is claimed is:

1. A method for treating sexual dysfumction, which comprises administering to an individual in need thereof a therapeutically effective amount of an active agent on an as-needed basis, wherein said active agent is selected from the group consisting of:
a. Substituted-benzyl or substituted-indolyl cyclic amino- substituted N-aryl or heteroaryl cyclic amines (illustrated below) as disclosed in U.S. Pat. No. 6,225,324 and salts, enantiomers, analogs, esters, amides, prodrugs, active metabolites, and derivatives thereof; 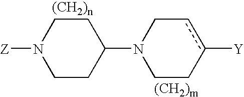
and/or hydrates thereof wherein
Z is selected from phenyl, benzodioxolone, benzodioxole, benzothiazole, pyridine, pyridazine, pyrimidine, and quinoline moieties that are unsubstituted or optimally substituted with one to three substituents selected from C₁₋₄ alkyl, C₁₋₄ alkoxy, cyano, and halo;
the solid and dotted lines denote either a double or a single covalent bond;
m and n are independently integers 1 to 3; and 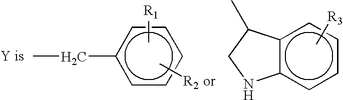
in which R₁ and R₂ are independently selected from hydrogen, halogen, and alkoxy, and R₃ is hydrogen, halogen, or cyano;
b. The compound shown below identified as BMS-296859; 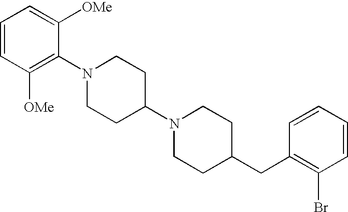
c. Thiophene and benzothiophene compounds (illustrated below) as disclosed in U.S. Pat. No. 6,262,056 and PCT Publication No. WO99/02516 and salts, enantiomers, analogs, esters, amides, prodrugs, active metabolites, and derivatives thereof; 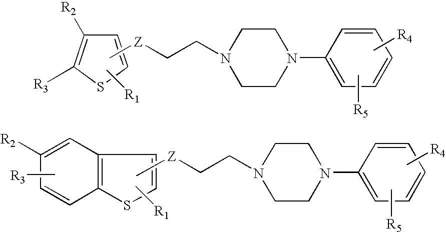
d. 3-[2-(1-(4′-piperonylpiperazinyl))indolyl]-carboxaldehydes (illustrated below) as disclosed in PCT Publication No. WO94/25454 and salts, enantiomers, analogs, esters, amides, prodrugs, active metabolites, and derivatives thereof; 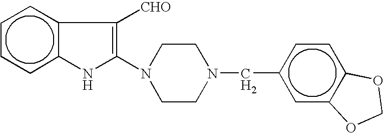
e. 3-[4-(3-substituted phenyl)piperazin-1-yl]-1-(benzo[b]thiophen-3-yl)propanol derivatives (illustrated below) as disclosed in Orus L et al. (2002) Pharmazie 57: 515-8 and salts, enantiomers, analogs, esters, amides, prodrugs, active metabolites, and derivatives thereof; 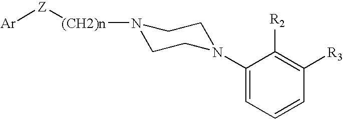
f. 1-aryl-3-[4-arylpiperazin-1-yl]-1-propane derivatives (illustrated below) as disclosed in Orus L et al. (2002) J Med Chem 45: 4128-39 and salts, enantiomers, analogs, esters, amides, prodrugs, active metabolites, and derivatives thereof; 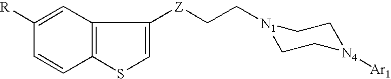
g. The compound shown below and salts, enantiomers, analogs, esters, amides, prodrugs, active metabolites, and derivatives thereof; 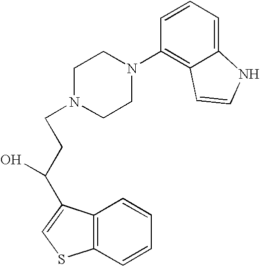
h. 3-[4-(aryl)piperazin-1-yl]-1-(benzo[b]thiophen-2-yl)propane derivatives (illustrated below) as disclosed in Orus L et al. (2002) Pharmazie 57: 355-7 and salts, enantiomers, analogs, esters, amides, prodrugs, active metabolites, and derivatives thereof; 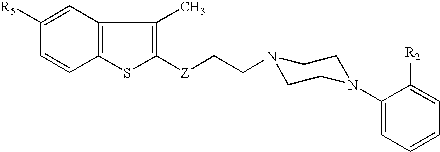
i. 1-aryl-3-(4-arylpiperazin-1-yl)propane derivatives (illustrated below) as disclosed in Martinez-Esparza J et al. (2001) J Med Chem 44: 418-28 and salts, enantiomers, analogs, esters, amides, prodrugs, active metabolites, and derivatives thereof; 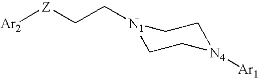
j. 3-[4-(aryl)piperazin-1-yl]-1-(benzo[b]thiophen-3-yl)propane derivatives (illustrated below) as disclosed in Martinez J et al. (2001) Eur J Med Chem 36: 55-61 and salts, enantiomers, analogs, esters, amides, prodrugs, active metabolites, and derivatives thereof; 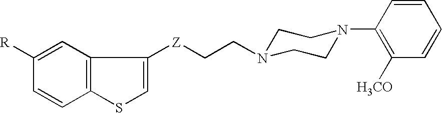
k. 3-[(4-aryl)piperazin-1-yl]-1-arylpropane derivatives (illustrated below) as disclosed in Oficialdegui A M et al. (2000) Farmaco 55: 345-53 and salts, enantiomers, analogs, esters, amides, prodrugs, active metabolites, and derivatives thereof; 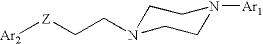
l. The compound VN2222 (illustrated below) as identified and disclosed in Tordera R M et al. (2002) Eur J Pharmacol 442: 63-71 and salts, enantiomers, analogs, esters, amides, prodrugs, active metabolites, and derivatives thereof; 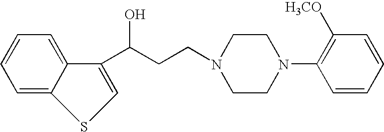
m. Arylpiperazinyl cyclohexyl derivatives (illustrated below) as disclosed in U.S. Pat. No. 6,465,482 and salts, enantiomers, analogs, esters, amides, prodrugs, active metabolites, and derivatives thereof; 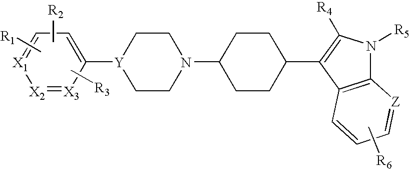
n. Aryl piperazinyl cyclohexyl derivatives (illustrated below) as disclosed in U.S. Pat. No. 6,337,336 and salts, enantiomers, analogs, esters, amides, prodrugs, active metabolites, and derivatives thereof; 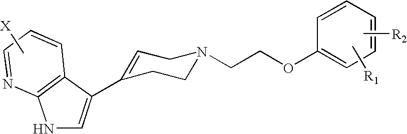
o. Arylpiperazinyl-cyclohexyl indole derivatives (illustrated below) as disclosed in U.S. Pat. No. 6,313,126 and salts, enantiomers, analogs, esters, amides, prodrugs, active metabolites, and derivatives thereof; 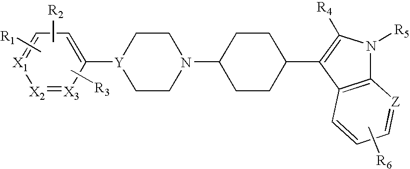
p. 3,4-Dihydro-2H-benzo[1,4]oxazinyl-methyl)-[3-(1 H-indol-3yl)-alkyl]-amines (illustrated below) as disclosed in U.S. Pat. No. 6,313,114 and salts, enantiomers, analogs, esters, amides, prodrugs, active metabolites, and derivatives thereof; 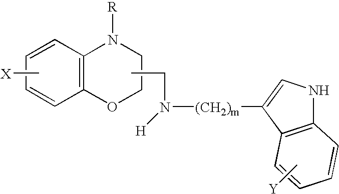
q. N-arloxyethyl-alkylamines (illustrated below) as disclosed in U.S. Pat. No. 6,291,683 and salts, enantiomers, analogs, esters, amides, prodrugs, active metabolites, and derivatives thereof; 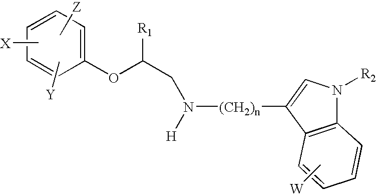
r. Tetrahydroisoquinolinyl-indole derivatives (illustrated below) as disclosed in U.S. Pat. No. 6,245,780 and salts, enantiomers, analogs, esters, amides, prodrugs, active metabolites, and derivatives thereof; 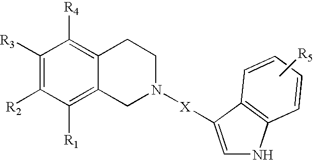
s. 3,4-Dihydro-2H-benzo[1,4]oxazine derivatives (illustrated below) as disclosed in U.S. Pat. No. 6,221,863 and salts, enantiomers, analogs, esters, amides, prodrugs, active metabolites, and derivatives thereof; 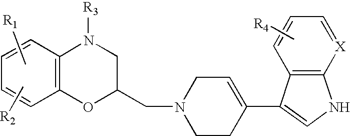
t. 1,4-disubstituted cyclohexane derivatives (illustrated below) as disclosed in U.S. Pat. No. 6,200,994 and salts, enantiomers, analogs, esters, amides, prodrugs, active metabolites, and derivatives thereof; 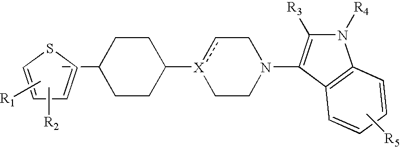
u. Indol-3-yl-cyclohexylamine derivatives (illustrated below) as disclosed in U.S. Pat. No. 6,162,803 and salts, enantiomers, analogs, esters, amides, prodrugs, active metabolites, and derivatives thereof; 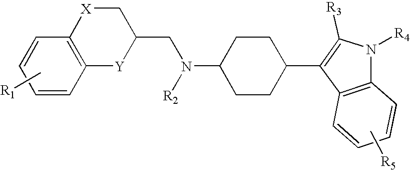
v. N-aryloxyethyl-indoly-alkylamines (illustrated below) as disclosed in U.S. Pat. No. 6,150,533 and salts, enantiomers, analogs, esters, amides, prodrugs, active metabolites, and derivatives thereof; 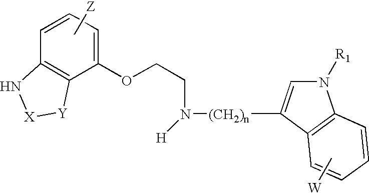
w. Aryloxyethyl-indoly-alkylamine derivatives (illustrated below) as disclosed in U.S. Pat. No. 6,121,307 and salts, enantiomers, analogs, esters, amides, prodrugs, active metabolites, and derivatives thereof; 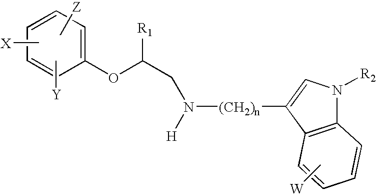
x. N-aryloxyethylarnine derivatives (illustrated below) as disclosed in U.S. Pat. No. 6,110,956 and salts, enantiomers, analogs, esters, amides, prodrugs, active metabolites, and derivatives thereof; 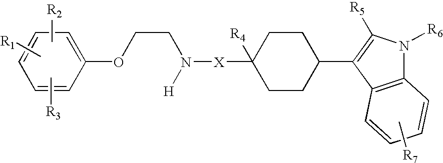
y. Aryl-8-azabicyclo[3.2.1]octanes (illustrated below) as disclosed in PCT Publication No. WO02/96906 and salts, enantiomers, analogs, esters, amides, prodrugs, active metabolites, and derivatives thereof; 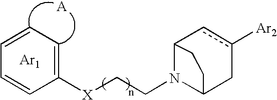
z. Azaindole derivatives (illustrated below) as disclosed in PCT Publication No. WO00/64898 and salts, enantiomers, analogs, esters, amides, prodrugs, active metabolites, and derivatives thereof; 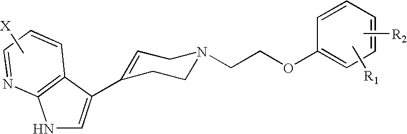
aa. Dihydroisoquinolinyl-indole derivatives (illustrated below) as disclosed in PCT Publication No. WO00/64886 and salts, enantiomers, analogs, esters, amides, prodrugs, active metabolites, and derivatives thereof; 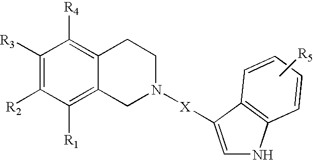
bb. 3,4-dihydro-2H-benzo[1,4]oxazine derivatives (illustrated below) as disclosed in PCT Publication No. WO0/40581 and salts, enantiomers, analogs, esters, amides, prodrugs, active metabolites, and derivatives thereof; 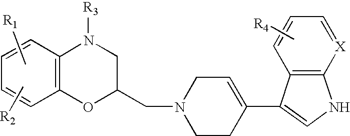
cc. 3,4-dihydro-2Hbenzo[1,4]oxazinyl-methyl)-[3-(1H-indoI-3-yI)-alkyI]amines (illustrated below) as disclosed in PCT Publication No. WO00/40580 and salts, enantiomers, analogs, esters, amides, prodrugs, active metabolites, and derivatives thereof; 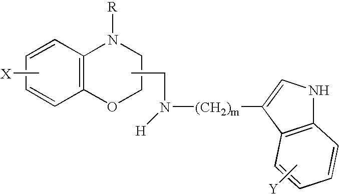
dd. 1,4 disubstituted cyclohexane derivatives (illustrated below) as disclosed in PCT Publication No. WO00/40579 and salts, enantiomers, analogs, esters, amides, prodrugs, active metabolites, and derivatives thereof; 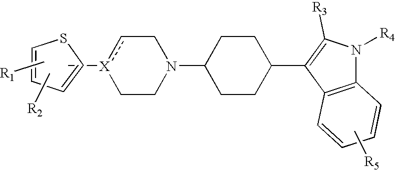
ee. Arylpiperazinyl cyclohexyl derivatives (illustrated below) as disclosed in PCT Publication No. WO00/40554 and salts, enantiomers, analogs, esters, amides, prodrugs, active metabolites, and derivatives thereof; 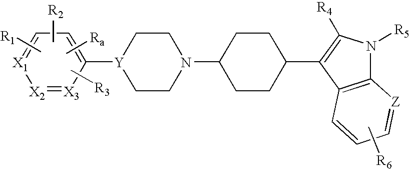
ff. Indol-3-yl-cyclohexylamine derivatives (illustrated below) as disclosed in PCT Publication No. WO99/51592 and salts, enantiomers, analogs, esters, amides, prodrugs, active metabolites, and derivatives thereof; 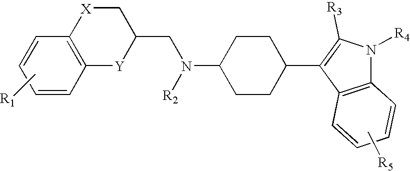
gg. N-aryloxyethyl-indoly-alkylamines (illustrated below) as disclosed in PCT Publication No. WO99/51591 and salts, enantiomers, analogs, esters, amides, prodrugs, active metabolites, and derivatives thereof; 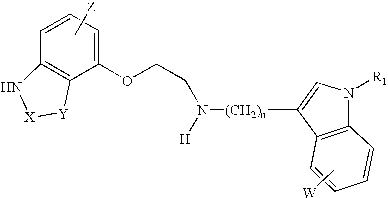
hh N-aryloxyethylamine derivatives (illustrated below) as disclosed in PCT Publication No. WO99/51576 and salts, enantiomers, analogs, esters, amides, prodrugs, active metabolites, and derivatives thereof; 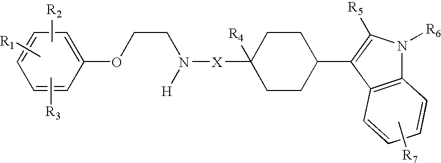
ii. Aryloxyethyl-indoly-alkylamine derivatives (illustrated below) as disclosed in PCT Publication No. WO99/51575 and salts, enantiomers, analogs, esters, amides, prodrugs, active metabolites, and derivatives thereof; 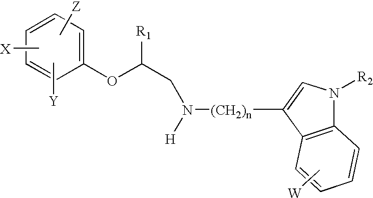
jj. Substituted phenoxypropylamines (illustrated below) as disclosed in U.S. Patent Application No. 2002/0111358 and PCT Publication No. WO 02/422297 and salts, enantiomers, analogs, esters, amides, prodrugs, active metabolites, and derivatives thereof; 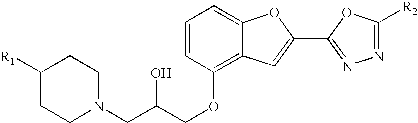
kk. Substituted aminothienopyridines (illustrated below) as disclosed in U.S. Pat. No. 5,252,581 and salts, enantiomers, analogs, esters, amides, prodrugs, active metabolites, and derivatives thereof; 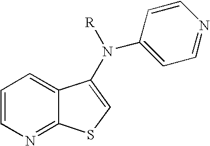
ll. Aromatic amines of arylpiperazines (illustrated below) as disclosed in PCT Publication No. WO 98/23590 and salts, enantiomers, analogs, esters, amides, prodrugs, active metabolites, and derivatives thereof; 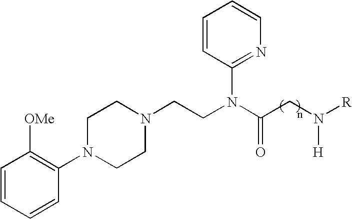
mm. Piperidines and pyrrolidines (illustrated below) as disclosed in PCT Publication No. WO 97/40038 and salts, enantiomers, analogs, esters, amides, prodrugs, active metabolites, and derivatives thereof; 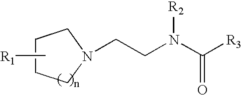
nn. The compound (+)-MCU-629 as shown below and salts, enantiomers, analogs, esters, amides, prodrugs, active metabolites, and derivatives thereof; 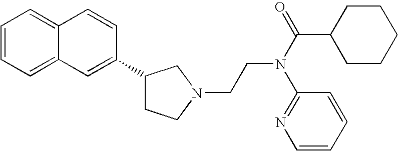
oo. Benzoxazinone derivatives (illustrated below) as disclosed in PCT Publication No. WO 03/091248 and salts, enantiomers, analogs, esters, amides, prodrugs, active metabolites, and derivatives thereof; 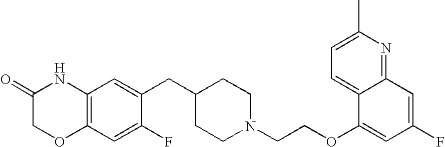
pp. Indole derivatives (illustrated below) as disclosed in PCT Publication WO 01/46181 and salts, enantiomers, analogs, esters, amides, prodrugs, active metabolites, and derivatives thereof; 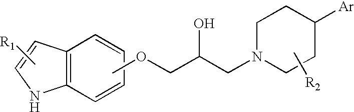
qq. The compound shown below and salts, enantiomers, analogs, esters, amides, prodrugs, active metabolites, and derivatives thereof; 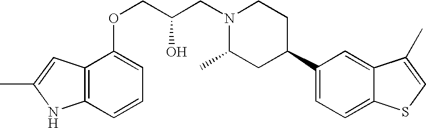
rr. Tetrahydropyridine and piperazine derivatives (illustrated below) as disclosed in U.S. Pat. Nos. 6,596,722, 6,476,035, and 6,391,882, U.S. Patent Application Nos. 2002/0035113, 2002/0173512, and 2003/0018050, and PCT Publication Nos. WO 00/43382, WO 99/05140, and WO 99/67237 and salts, enantiomers, analogs, esters, amides, prodrugs, active metabolites, and derivatives thereof; and 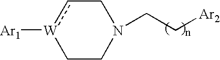
ss. The compound LU-36-274 as shown below and salts, enantiomers, analogs, esters, amides, prodrugs, active metabolites, and derivatives thereof.

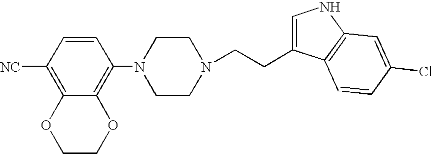

2. The method of claim 1, wherein the sexual dysfunction is Premature Ejaculation.

3. The method of claim 1, wherein the active agent is administered from about 0 minutes to about 10 hours prior to commencement of an activity wherein suppression of the symptoms of sexual dysfumction would be desirable.

4. The method of claim 3, wherein the active agent is administered from about from about 0 minutes to about 6 hours prior to commencement of an activity wherein suppression of the symptoms of sexual dysfunction would be desirable.

5. The method of claim 3, wherein the active agent is administered from about 0 minutes to about 4 hours prior to commencement of an activity wherein suppression of the symptoms of sexual dysfunction would be desirable.

6. The method of claim 1, wherein the active agent is contained within a pharmaceutical formulation.

7. The method of claim 6, wherein the pharmaceutical formulation is a unit dosage form.

8. The method of claim 6, wherein the pharmaceutical formulation is a controlled release dosage form.

9. The method of claim 6, wherein the pharmaceutical formulation is a delayed release dosage form.

10. The method of claim 1, wherein the active agent is administered by a mode selected from the group consisting of oral, transmucosal, topical, transdermal, and parenteral.

11. The method of claim 10, wherein the active agent is administered transmucosally.

12. The method of claim 11, wherein the mode of transmucosal delivery of the active agent is selected from the group consisting of sublingual, buccal, intranasal, transurethral, rectal, and inhalation.

13. The method of claim 10, wherein the active agent is administered orally.

14. The method of claim 6, wherein the active agent is administered orally.

15. The method of claim 14, wherein the pharmaceutical formulation is selected from the group consisting of tablets, capsules, caplets, solutions, suspensions, syrups, granules, beads, powders, pellets, and rapidly disintegrating tablets.

16. The method of claim 15, wherein the rapidly disintegrating tablet is an effervescent tablet.

17. The method of claim 15, wherein the pharmaceutical formulation comprises a tablet.

18. The method of claim 15, wherein the pharmaceutical formulation comprises a capsule.

19. The method of claim 6, wherein the pharmaceutical formulation further comprises an additional active agent.

20. The method of claim 1, wherein the active agent is a compound selected from the group consisting of:

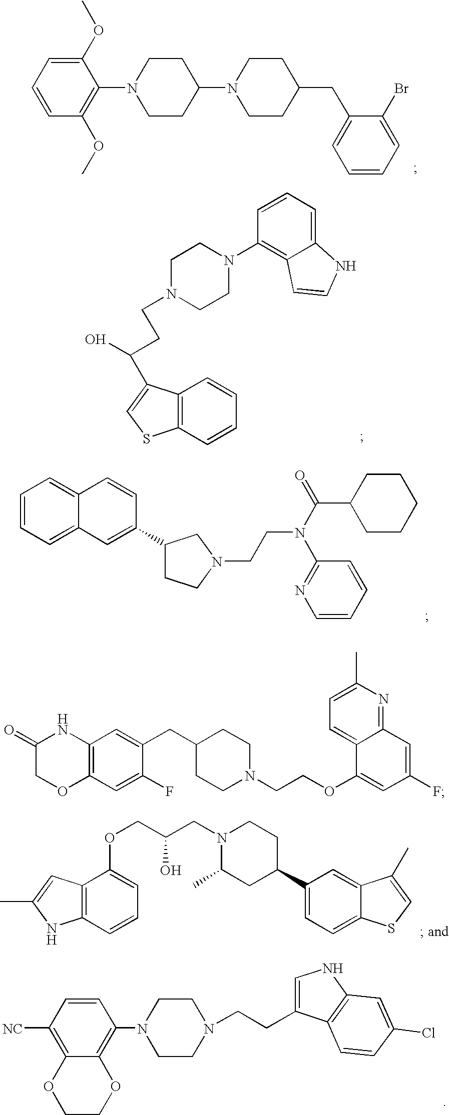

21. The method of claim 1, wherein the active agent comprises the following compound

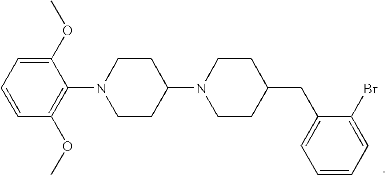

22. A pharmaceutical formulation for treating sexual dysfunction, which comprises administering to an individual in need thereof a therapeutically effective amount of an active agent on an as-needed basis, wherein said active agent is selected from the group consisting of:
a. Substituted-benzyl or substituted-indolyl cyclic amino- substituted N-aryl or heteroaryl cyclic amines (illustrated below) as disclosed in U.S. Pat. No. 6,225,324 and salts, enantiomers, analogs, esters, amides, prodrugs, active metabolites, and derivatives thereof; 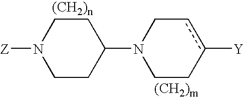
and/or hydrates thereof wherein
Z is selected from phenyl, benzodioxolone, benzodioxole, benzothiazole, pyridine, pyridazine, pyrimidine, and quinoline moieties that are unsubstituted or optimally substituted with one to three substituents selected from C₁₋₄ alkyl, C₁₋₄ alkoxy, cyano, and halo;
the solid and dotted lines denote either a double or a single covalent bond;
m and n are independently integers 1 to 3; and 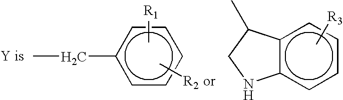
in which R₁ and R₂ are independently selected from hydrogen, halogen, and alkoxy, and R₃ is hydrogen, halogen, or cyano;
b. The compound shown below identified as BMS-296859; 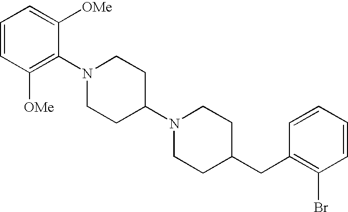
c. Thiophene and benzothiophene compounds (illustrated below) as disclosed in U.S. Pat. No. 6,262,056 and PCT Publication No. WO99/02516 and salts, enantiomers, analogs, esters, amides, prodrugs, active metabolites, and derivatives thereof; 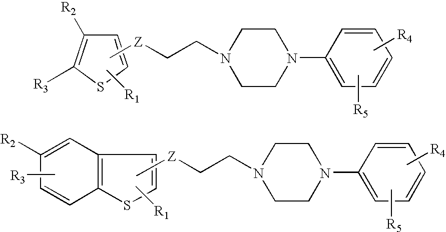
d. 3-[2-(1-(4′-piperonylpiperazinyl))indolyl]-carboxaldehydes (illustrated below) as disclosed in PCT Publication No. WO94/25454 and salts, enantiomers, analogs, esters, amides, prodrugs, active metabolites, and derivatives thereof; 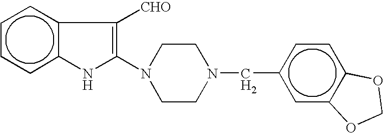
e. 3-[4-(3-substituted phenyl)piperazin-1-yl]-1-(benzo[b]thiophen-3-yl)propanol derivatives (illustrated below) as disclosed in Orus L et al. (2002) Pharmazie 57: 515-8 and salts, enantiomers, analogs, esters, amides, prodrugs, active metabolites, and derivatives thereof; 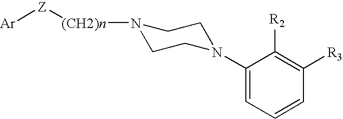
f. 1-aryl-3-[4-arylpiperazin-1-yl]-1-propane derivatives (illustrated below) as disclosed in Orus L et al. (2002) J Med Chem 45: 4128-39 and salts, enantiomers, analogs, esters, amides, prodrugs, active metabolites, and derivatives thereof; 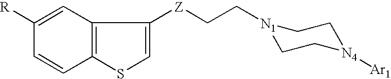
g. The compound shown below and salts, enantiomers, analogs, esters, amides, prodrugs, active metabolites, and derivatives thereof; 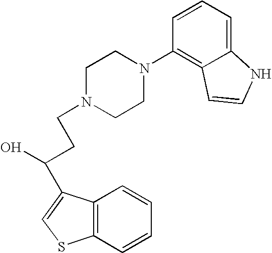
h. 3-[4-(aryl)piperazin-1-yl]-1-(benzo[b]thiophen-2-yl)propane derivatives (illustrated below) as disclosed in Orus L et al. (2002) Pharmazie 57: 355-7 and salts, enantiomers, analogs, esters, amides, prodrugs, active metabolites, and derivatives thereof; 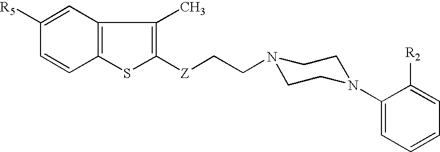
i. 1-aryl-3-(4-arylpiperazin-1-yl)propane derivatives (illustrated below) as disclosed in Martinez-Esparza J et al. (2001) J Med Chem 44: 418-28 and salts, enantiomers, analogs, esters, amides, prodrugs, active metabolites, and derivatives thereof; 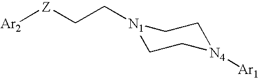
j. 3-[4-(aryl)piperazin-1-yl]-1-(benzo[b]thiophen-3-yl)propane derivatives (illustrated below) as disclosed in Martinez J et al. (2001) Eur J Med Chem 36: 55-61 and salts, enantiomers, analogs, esters, amides, prodrugs, active metabolites, and derivatives thereof; 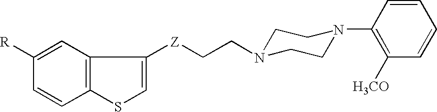
k. 3-[(4-aryl)piperazin-1-yl]-1-arylpropane derivatives (illustrated below) as disclosed in Oficialdegui A M et al. (2000) Farmaco 55: 345-53 and salts, enantiomers, analogs, esters, amides, prodrugs, active metabolites, and derivatives thereof; 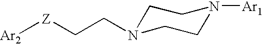
1. The compound VN2222 (illustrated below) as identified and disclosed in Tordera R M et al. (2002) Eur J Pharmacol 442: 63-71 and salts, enantiomers, analogs, esters, amides, prodrugs, active metabolites, and derivatives thereof; 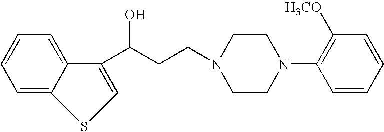
m. Arylpiperazinyl cyclohexyl derivatives (illustrated below) as disclosed in U.S. Pat. No. 6,465,482 and salts, enantiomers, analogs, esters, amides, prodrugs, active metabolites, and derivatives thereof; 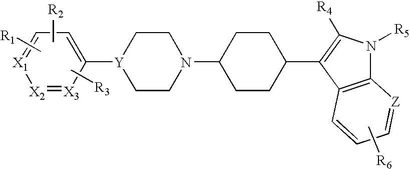
n. Aryl piperazinyl cyclohexyl derivatives (illustrated below) as disclosed in U.S. Pat. No. 6,337,336 and salts, enantiomers, analogs, esters, amides, prodrugs, active metabolites, and derivatives thereof; 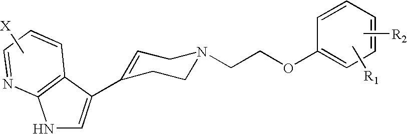
o. Arylpiperazinyl-cyclohexyl indole derivatives (illustrated below) as disclosed in U.S. Pat. No. 6,313,126 and salts, enantiomers, analogs, esters, amides, prodrugs, active metabolites, and derivatives thereof; 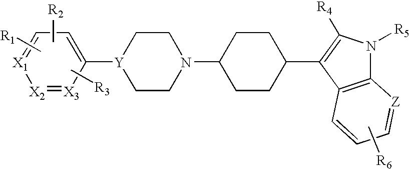
p. 3,4-Dihydro-2H-benzo[1,4]oxazinyl-methyl)-[3-(1H-indol-3yl)-alkyl]-amines (illustrated below) as disclosed in U.S. Pat. No. 6,313,114 and salts, enantiomers, analogs, esters, amides, prodrugs, active metabolites, and derivatives thereof; 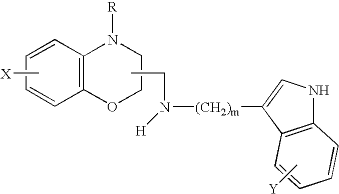
q. N-arloxyethyl-alkylamines (illustrated below) as disclosed in U.S. Pat. No. 6,291,683 and salts, enantiomers, analogs, esters, amides, prodrugs, active metabolites, and derivatives thereof; 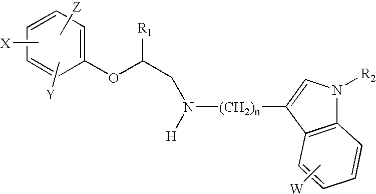
r. Tetrahydroisoquinolinyl-indole derivatives (illustrated below) as disclosed in U.S. Pat. No. 6,245,780 and salts, enantiomers, analogs, esters, amides, prodrugs, active metabolites, and derivatives thereof; 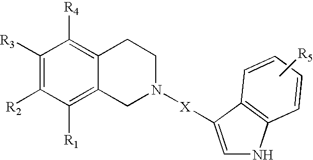
s. 3,4-Dihydro-2H-benzo[1,4]oxazine derivatives (illustrated below) as disclosed in U.S. Pat. No. 6,221,863 and salts, enantiomers, analogs, esters, amides, prodrugs, active metabolites, and derivatives thereof; 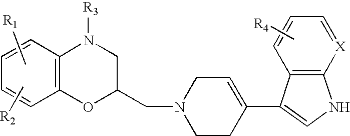
t. 1,4-disubstituted cyclohexane derivatives (illustrated below) as disclosed in U.S. Pat. No. 6,200,994 and salts, enantiomers, analogs, esters, amides, prodrugs, active metabolites, and derivatives thereof; 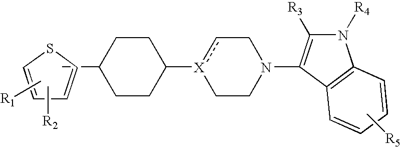
u. Indol-3-yl-cyclohexylamine derivatives (illustrated below) as disclosed in U.S. Pat. No. 6,162,803 and salts, enantiomers, analogs, esters, amides, prodrugs, active metabolites, and derivatives thereof; 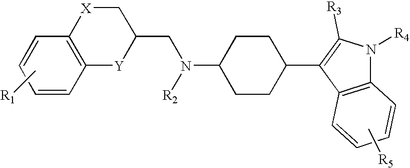
v. N-aryloxyethyl-indoly-alkylamines (illustrated below) as disclosed in U.S. Pat. No. 6,150,533 and salts, enantiomers, analogs, esters, amides, prodrugs, active metabolites, and derivatives thereof; 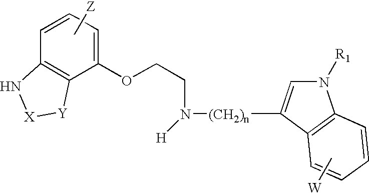
w. Aryloxyethyl-indoly-alkylamine derivatives (illustrated below) as disclosed in U.S. Pat. No. 6,121,307 and salts, enantiomers, analogs, esters, amides, prodrugs, active metabolites, and derivatives thereof; 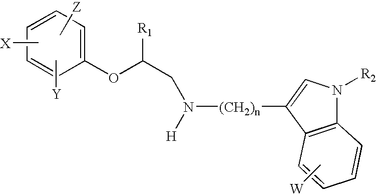
x. N-aryloxyethylarnine derivatives (illustrated below) as disclosed in U.S. Pat. No. 6,110,956 and salts, enantiomers, analogs, esters, amides, prodrugs, active metabolites, and derivatives thereof; 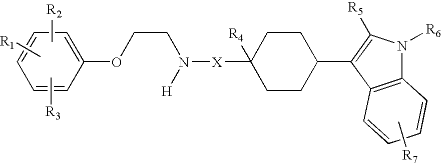
y. Aryl-8-azabicyclo[3.2.1]octanes (illustrated below) as disclosed in PCT Publication No. WO02/96906 and salts, enantiomers, analogs, esters, amides, prodrugs, active metabolites, and derivatives thereof; 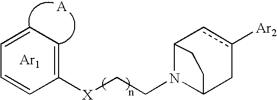
z. Azaindole derivatives (illustrated below) as disclosed in PCT Publication No. WO00/64898 and salts, enantiomers, analogs, esters, amides, prodrugs, active metabolites, and derivatives thereof; 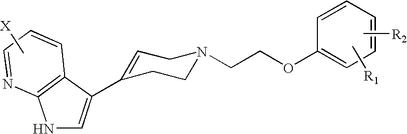
aa. Dihydroisoquinolinyl-indole derivatives (illustrated below) as disclosed in PCT Publication No. WO00/64886 and salts, enantiomers, analogs, esters, amides, prodrugs, active metabolites, and derivatives thereof; 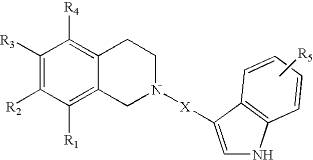
bb. 3,4-dihydro-2H-benzo[1,4]oxazine derivatives (illustrated below) as disclosed in PCT Publication No. WO00/40581 and salts, enantiomers, analogs, esters, amides, prodrugs, active metabolites, and derivatives thereof; 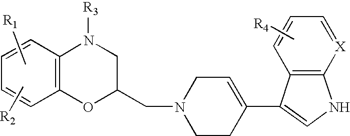
cc. 3,4-dihydro-2Hbenzo[1,4]oxazinyl-methyl)-[3-(1H-indoI-3-yI)-alkyI]amines (illustrated below) as disclosed in PCT Publication No. WO00/40580 and salts, enantiomers, analogs, esters, amides, prodrugs, active metabolites, and derivatives thereof; 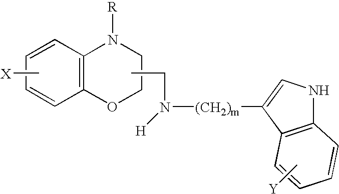
dd. 1,4disubstituted cyclohexane derivatives (illustrated below) as disclosed in PCT Publication No. WO00/40579 and salts, enantiomers, analogs, esters, amides, prodrugs, active metabolites, and derivatives thereof; 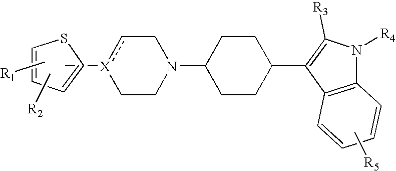
ee. Arylpiperazinyl cyclohexyl derivatives (illustrated below) as disclosed in PCT Publication No. WO00/40554 and salts, enantiomers, analogs, esters, amides, prodrugs, active metabolites, and derivatives thereof; 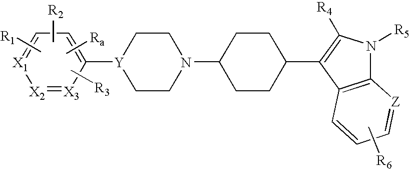
ff. Indol-3-yl-cyclohexylamine derivatives (illustrated below) as disclosed in PCT Publication No. WO99/51592 and salts, enantiomers, analogs, esters, amides, prodrugs, active metabolites, and derivatives thereof; 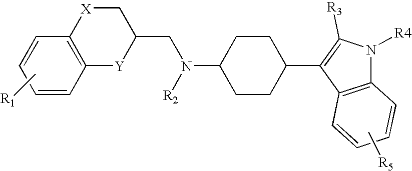
gg. N-aryloxyethyl-indoly-alkylamines (illustrated below) as disclosed in PCT Publication No. WO99/51591 and salts, enantiomers, analogs, esters, amides, prodrugs, active metabolites, and derivatives thereof; 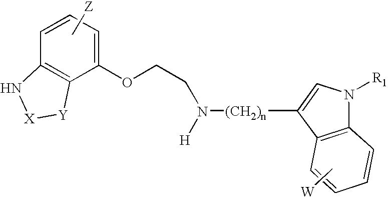
hh. N-aryloxyethylamine derivatives (illustrated below) as disclosed in PCT Publication No. WO99/51576 and salts, enantiomers, analogs, esters, amides, prodrugs, active metabolites, and derivatives thereof; 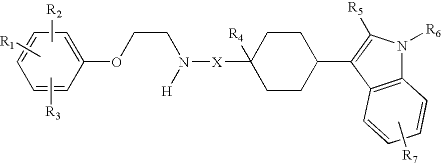
ii. Aryloxyethyl-indoly-alkylamine derivatives (illustrated below) as disclosed in PCT Publication No. WO99/51575 and salts, enantiomers, analogs, esters, amides, prodrugs, active metabolites, and derivatives thereof; 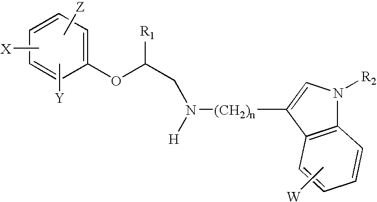
jj. Substituted phenoxypropylamines (illustrated below) as disclosed in U.S. Patent Application No. 2002/0111358 and PCT Publication No. WO 02/422297 and salts, enantiomers, analogs, esters, amides, prodrugs, active metabolites, and derivatives thereof; 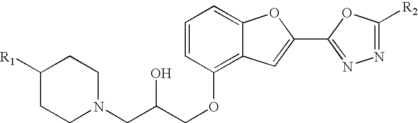
kk. Substituted aminothienopyridines (illustrated below) as disclosed in U.S. Pat. No. 5,252,581 and salts, enantiomers, analogs, esters, amides, prodrugs, active metabolites, and derivatives thereof; 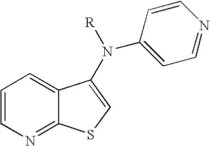
ll. Aromatic amines of arylpiperazines (illustrated below) as disclosed in PCT Publication No. WO 98/23590 and salts, enantiomers, analogs, esters, amides, prodrugs, active metabolites, and derivatives thereof; 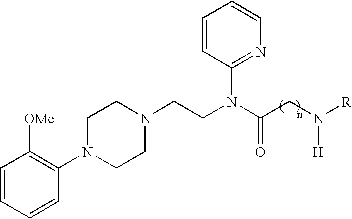
mm. Piperidines and pyrrolidines (illustrated below) as disclosed in PCT Publication No. WO 97/40038 and salts, enantiomers, analogs, esters, amides, prodrugs, active metabolites, and derivatives thereof; 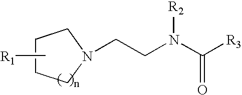
nn. The compound (+)-MCU-629 as shown below and salts, enantiomers, analogs, esters, amides, prodrugs, active metabolites, and derivatives thereof; 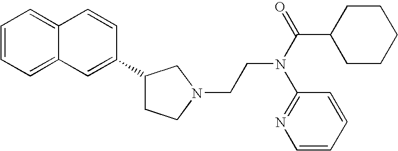
oo. Benzoxazinone derivatives (illustrated below) as disclosed in PCT Publication No. WO 03/091248 and salts, enantiomers, analogs, esters, amides, prodrugs, active metabolites, and derivatives thereof; 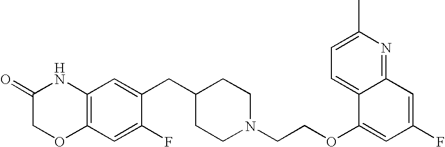
pp. Indole derivatives (illustrated below) as disclosed in PCT Publication WO 01/46181 and salts, enantiomers, analogs, esters, amides, prodrugs, active metabolites, and derivatives thereof; 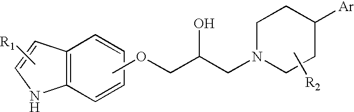
qq. The compound shown below and salts, enantiomers, analogs, esters, amides, prodrugs, active metabolites, and derivatives thereof; 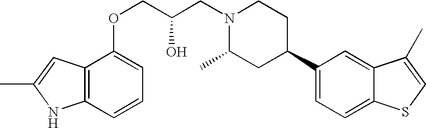
rr. Tetrahydropyridine and piperazine derivatives (illustrated below) as disclosed in U.S. Pat. Nos. 6,596,722, 6,476,035, and 6,391,882, U.S. Patent Application Nos. 2002/0035113, 2002/0173512, and 2003/0018050, and PCT Publication Nos. WO 00/43382, WO 99/05140, and WO 99/67237 and salts, enantiomers, analogs, esters, amides, prodrugs, active metabolites, and derivatives thereof; and 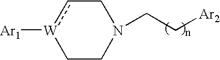
ss. The compound LU-36-274 as shown below and salts, enantiomers, analogs, esters, amides, prodrugs, active metabolites, and derivatives thereof.

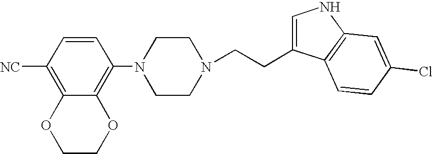

23. The pharmaceutical formulation of claim 22, further comprising a carrier suitable for transmucosal drug delivery buccally, sublingually, intranasally, rectally, or by inhalation.

24. The pharmaceutical formulation of claim 22, wherein the sexual dysfunction is Premature Ejaculation.

25. The pharmaceutical formulation of claim 23, comprising a solid dosage form for application to the buccal mucosa, and wherein the carrier is suitable for buccal drug delivery.

26. The pharmaceutical formulation of claim 25, wherein the carrier is a hydrolyzable polymer.

27. The pharmaceutical formulation of claim 25, wherein the dosage form further comprises an adhesive suitable for affixing the dosage form to the buccal mucosa.

28. The pharmaceutical formulation of claim 23, comprising a dosage form for application to the sublingual mucosa, and wherein the carrier is suitable for sublingual drug delivery.

29. The pharmaceutical formulation of claim 23, comprising a dosage form for application to the rectal mucosa, and wherein the carrier is suitable for rectal drug delivery.

30. The pharmaceutical formulation of claim 29, comprising a rectal suppository.

31. The pharmaceutical formulation of claim 23, comprising a dosage form suitable for inhalation.

32. The pharmaceutical formulation of claim 31, comprising a liquid.

33. The pharmaceutical formulation of claim 31, comprising a dry powder.

34. The pharmaceutical formulation of claim 31 comprising an aerosol composition.

35. The pharmaceutical formulation of claim 23, wherein the pharmaceutical formulation further comprises an additional active agent.

36. The pharmaceutical formulation of claim 22, wherein the active agent is a compound selected from the group consisting of:

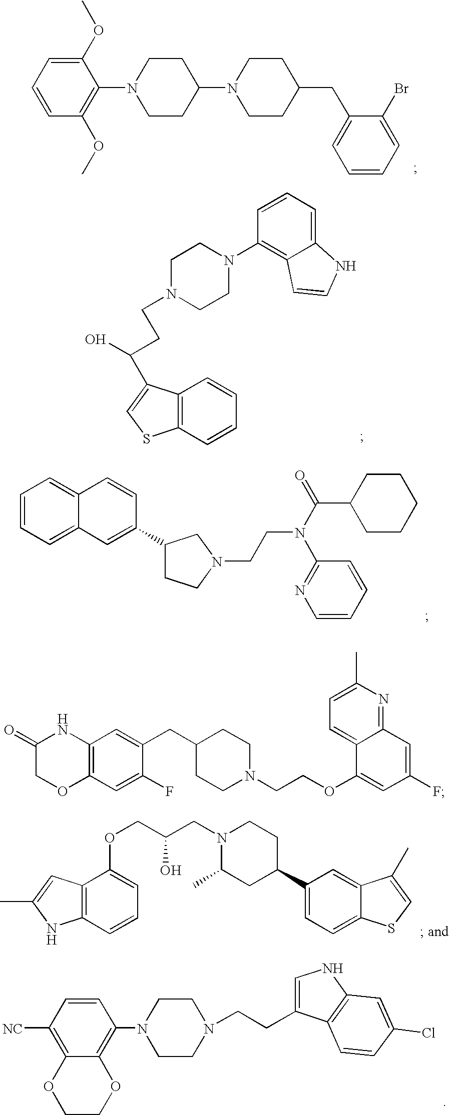

37. The pharmaceutical formulation of claim 36, further comprising a carrier suitable for transmucosal drug delivery bucally, sublingually, intranasally, rectally, or by inhalation.

38. The pharmaceutical formulation of claim 22, wherein the active agent comprises the following compound

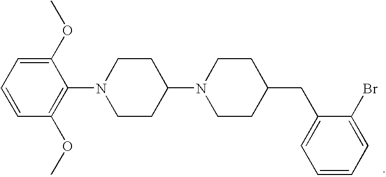

39. The pharmaceutical formulation of claim 38, further comprising a carrier suitable for transmucosal drug delivery bucally, sublingually, intranasally, rectally, or by inhalation.

40. A packaged kit for use in the treatment of sexual dysfunction on an as-needed basis, comprising: a pharmaceutical formulation of an active agent; a container housing the pharmaceutical formulation during storage and prior to administration; and instructions for carrying out drug administration in a manner effective to treat sexual dysfunction; wherein said active agent is selected from the group consisting of:
a. Substituted-benzyl or substituted-indolyl cyclic amino-substituted N-aryl or heteroaryl cyclic amines (illustrated below) as disclosed in U.S. Pat. No. 6,225,324 and salts, enantiomers, analogs, esters, amides, prodrugs, active metabolites, and derivatives thereof; 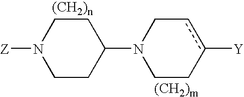
and/or hydrates thereof wherein
Z is selected from phenyl, benzodioxolone, benzodioxole, benzothiazole, pyridine, pyridazine, pyrimidine, and quinoline moieties that are unsubstituted or optimally substituted with one to three substituents selected from C₁₋₄ alkyl, C₁₋₄ alkoxy, cyano, and halo;
the solid and dotted lines denote either a double or a single covalent bond;
m and n are independently integers 1 to 3; and 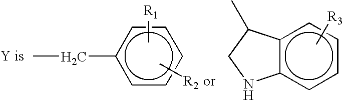
in which R₁ and R₂ are independently selected from hydrogen, halogen, and alkoxy, and R₃ is hydrogen, halogen, or cyano;
b. The compound shown below identified as BMS-296859; 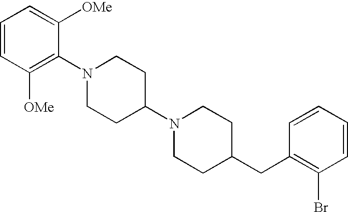
c. Thiophene and benzothiophene compounds (illustrated below) as disclosed in U.S. Pat. No. 6,262,056 and PCT Publication No. WO99/02516 and salts, enantiomers, analogs, esters, amides, prodrugs, active metabolites, and derivatives thereof; 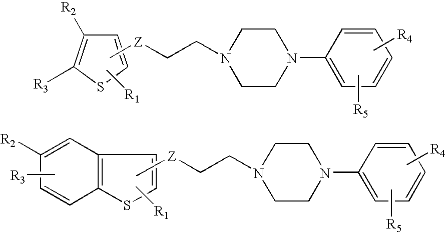
d. 3-[2-(1-(4′-piperonylpiperazinyl))indolyl]-carboxaldehydes (illustrated below) as disclosed in PCT Publication No. WO94/25454 and salts, enantiomers, analogs, esters, amides, prodrugs, active metabolites, and derivatives thereof; 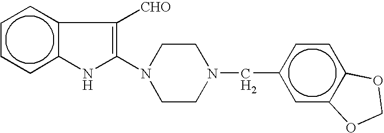
e. 3-[4-(3-substituted phenyl)piperazin-1-yl]-1-(benzo[b]thiophen-3-yl)propanol derivatives (illustrated below) as disclosed in Orus L et al. (2002) Pharmazie 57: 515-8 and salts, enantiomers, analogs, esters, amides, prodrugs, active metabolites, and derivatives thereof; 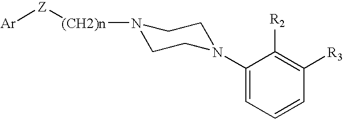
f. 1-aryl-3-[4-arylpiperazin-1-yl]-1-propane derivatives (illustrated below) as disclosed in Orus L et al. (2002) J Med Chem 45: 4128-39 and salts, enantiomers, analogs, esters, amides, prodrugs, active metabolites, and derivatives thereof; 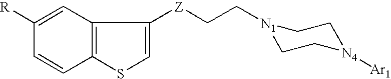
g. The compound shown below and salts, enantiomers, analogs, esters, amides, prodrugs, active metabolites, and derivatives thereof; 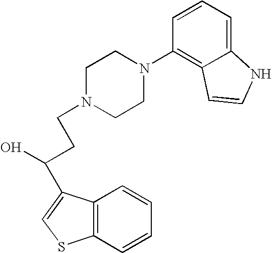
h. 3-[4-(aryl)piperazin-1-yl]-1-(benzo[b]thiophen-2-yl)propane derivatives (illustrated below) as disclosed in Orus L et al. (2002) Pharmazie 57: 355-7 and salts, enantiomers, analogs, esters, amides, prodrugs, active metabolites, and derivatives thereof; 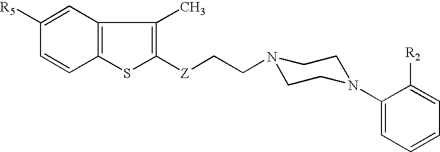
i. 1-aryl-3-(4-arylpiperazin-1-yl)propane derivatives (illustrated below) as disclosed in Martinez-Esparza J et al. (2001) J Med Chem 44: 418-28 and salts, enantiomers, analogs, esters, amides, prodrugs, active metabolites, and derivatives thereof; 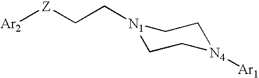
j. 3-[4-(aryl)piperazin-1-yl]-1-(benzo[b]thiophen-3-yl)propane derivatives (illustrated below) as disclosed in Martinez J et al. (2001) Eur J Med Chem 36: 55-61 and salts, enantiomers, analogs, esters, amides, prodrugs, active metabolites, and derivatives thereof; 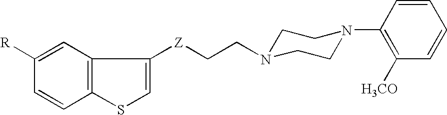
k. 3-[(4-aryl)piperazin-1-yl]-1-arylpropane derivatives (illustrated below) as disclosed in Oficialdegui A M et al. (2000) Farmaco 55: 345-53 and salts, enantiomers, analogs, esters, amides, prodrugs, active metabolites, and derivatives thereof; 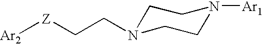
1. The compound VN2222 (illustrated below) as identified and disclosed in Tordera R M et al. (2002) Eur J Pharmacol 442: 63-71 and salts, enantiomers, analogs, esters, amides, prodrugs, active metabolites, and derivatives thereof; 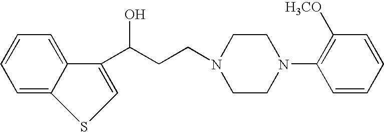
m. Arylpiperazinyl cyclohexyl derivatives (illustrated below) as disclosed in U.S. Pat. No. 6,465,482 and salts, enantiomers, analogs, esters, amides, prodrugs, active metabolites, and derivatives thereof; 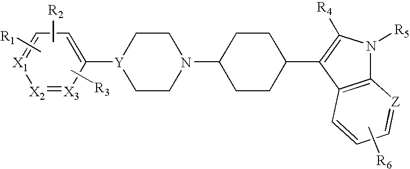
n. Aryl piperazinyl cyclohexyl derivatives (illustrated below) as disclosed in U.S. Pat. No. 6,337,336 and salts, enantiomers, analogs, esters, amides, prodrugs, active metabolites, and derivatives thereof; 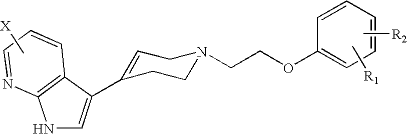
o. Arylpiperazinyl-cyclohexyl indole derivatives (illustrated below) as disclosed in U.S. Pat. No. 6,313,126 and salts, enantiomers, analogs, esters, amides, prodrugs, active metabolites, and derivatives thereof; 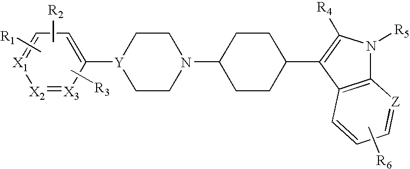
p. 3,4-Dihydro-2H-benzo[1,4]oxazinyl-methyl)-[3-(1H-indol-3yl)-alkyl]-amines (illustrated below) as disclosed in U.S. Pat. No. 6,313,114 and salts, enantiomers, analogs, esters, amides, prodrugs, active metabolites, and derivatives thereof; 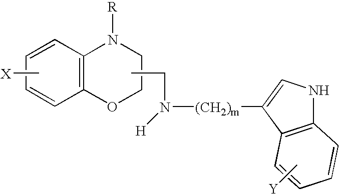
q. N-arloxyethyl-alkylamines (illustrated below) as disclosed in U.S. Pat. No. 6,291,683 and salts, enantiomers, analogs, esters, amides, prodrugs, active metabolites, and derivatives thereof; 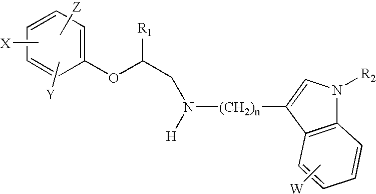
r. Tetrahydroisoquinolinyl-indole derivatives (illustrated below) as disclosed in U.S. Pat. No. 6,245,780 and salts, enantiomers, analogs, esters, amides, prodrugs, active metabolites, and derivatives thereof; 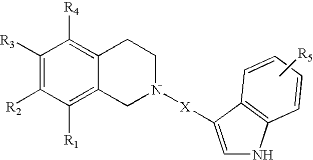
s. 3,4-Dihydro-2H-benzo[1,4]oxazine derivatives (illustrated below) as disclosed in U.S. Pat. No. 6,221,863 and salts, enantiomers, analogs, esters, amides, prodrugs, active metabolites, and derivatives thereof; 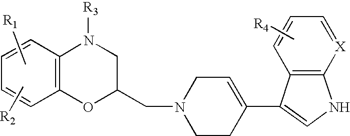
t. 1,4-disubstituted cyclohexane derivatives (illustrated below) as disclosed in U.S. Pat. No. 6,200,994 and salts, enantiomers, analogs, esters, amides, prodrugs, active metabolites, and derivatives thereof; 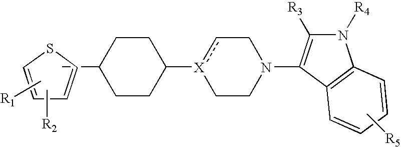
u. Indol-3-yl-cyclohexylamine derivatives (illustrated below) as disclosed in U.S. Pat. No. 6,162,803 and salts, enantiomers, analogs, esters, amides, prodrugs, active metabolites, and derivatives thereof; 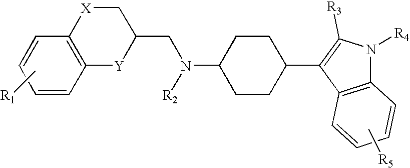
v. N-aryloxyethyl-indoly-alkylamines (illustrated below) as disclosed in U.S. Pat. No. 6,150,533 and salts, enantiomers, analogs, esters, amides, prodrugs, active metabolites, and derivatives thereof; 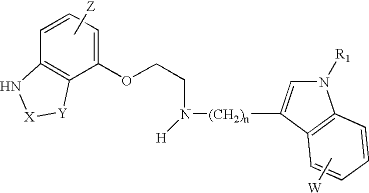
w. Aryloxyethyl-indoly-alkylamine derivatives (illustrated below) as disclosed in U.S. Pat. No. 6,121,307 and salts, enantiomers, analogs, esters, amides, prodrugs, active metabolites, and derivatives thereof; 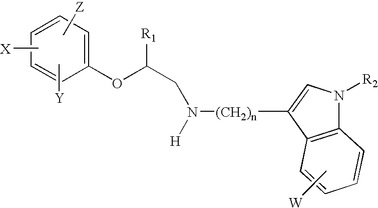
x. N-aryloxyethylarnine derivatives (illustrated below) as disclosed in U.S. Pat. No. 6,110,956 and salts, enantiomers, analogs, esters, amides, prodrugs, active metabolites, and derivatives thereof; 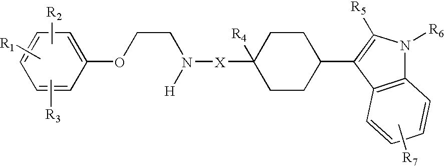
y. Aryl-8-azabicyclo[3.2.1]octanes (illustrated below) as disclosed in PCT Publication No. WO02/96906 and salts, enantiomers, analogs, esters, amides, prodrugs, active metabolites, and derivatives thereof; 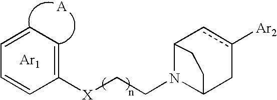
z. Azaindole derivatives (illustrated below) as disclosed in PCT Publication No. WO00/64898 and salts, enantiomers, analogs, esters, amides, prodrugs, active metabolites, and derivatives thereof; 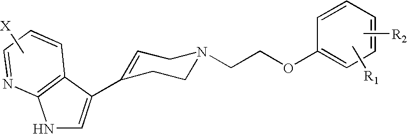
aa. Dihydroisoquinolinyl-indole derivatives (illustrated below) as disclosed in PCT Publication No. WO00/64886 and salts, enantiomers, analogs, esters, amides, prodrugs, active metabolites, and derivatives thereof; 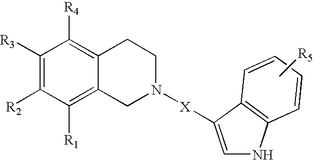
bb. 3,4-dihydro-2H-benzo[1,4]oxazine derivatives (illustrated below) as disclosed in PCT Publication No. WO00/40581 and salts, enantiomers, analogs, esters, amides, prodrugs, active metabolites, and derivatives thereof; 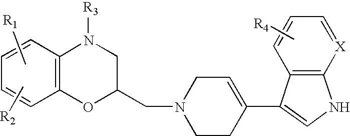
cc. 3,4-dihydro-2Hbenzo[1,4]oxazinyl-methyl)-[3-(1H-indoI-3-yI)-alkyI]amines (illustrated below) as disclosed in PCT Publication No. WO00/40580 and salts, enantiomers, analogs, esters, amides, prodrugs, active metabolites, and derivatives thereof; 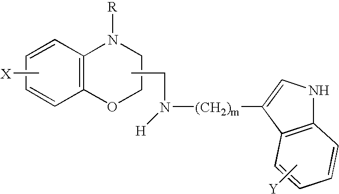
dd. 1,4 disubstituted cyclohexane derivatives (illustrated below) as disclosed in PCT Publication No. WO00/40579 and salts, enantiomers, analogs, esters, amides, prodrugs, active metabolites, and derivatives thereof; 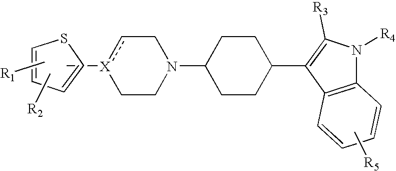
ee. Arylpiperazinyl cyclohexyl derivatives (illustrated below) as disclosed in PCT Publication No. WO00/40554 and salts, enantiomers, analogs, esters, amides, prodrugs, active metabolites, and derivatives thereof; 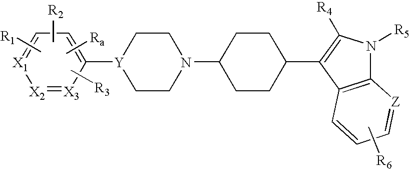
ff. Indol-3-yl-cyclohexylamine derivatives (illustrated below) as disclosed in PCT Publication No. WO99/51592 and salts, enantiomers, analogs, esters, amides, prodrugs, active metabolites, and derivatives thereof; 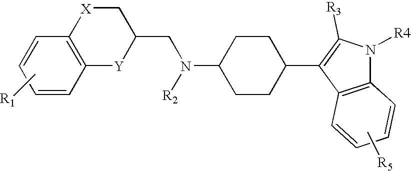
gg. N-aryloxyethyl-indoly-alkylamines (illustrated below) as disclosed in PCT Publication No. WO99/51591 and salts, enantiomers, analogs, esters, amides, prodrugs, active metabolites, and derivatives thereof; 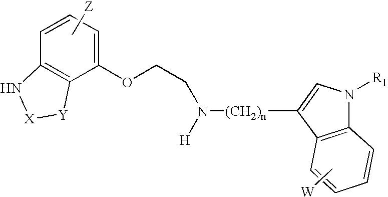
hh. N-aryloxyethylamine derivatives (illustrated below) as disclosed in PCT Publication No. WO99/51576 and salts, enantiomers, analogs, esters, amides, prodrugs, active metabolites, and derivatives thereof; 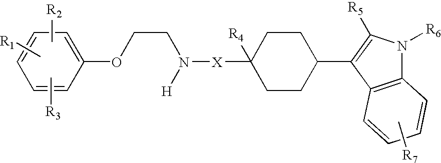
ii. Aryloxyethyl-indoly-alkylamine derivatives (illustrated below) as disclosed in PCT Publication No. WO99/51575 and salts, enantiomers, analogs, esters, amides, prodrugs, active metabolites, and derivatives thereof; 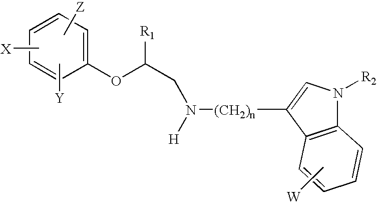
jj. Substituted phenoxypropylamines (illustrated below) as disclosed in U.S. Patent Application No. 2002/0111358 and PCT Publication No. WO 02/422297 and salts, enantiomers, analogs, esters, amides, prodrugs, active metabolites, and derivatives thereof; 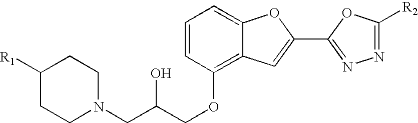
kk. Substituted aminothienopyridines (illustrated below) as disclosed in U.S. Pat. No. 5,252,581 and salts, enantiomers, analogs, esters, amides, prodrugs, active metabolites, and derivatives thereof; 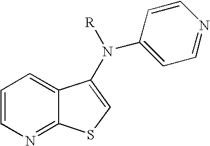
ll. Aromatic amines of arylpiperazines (illustrated below) as disclosed in PCT Publication No. WO 98/23590 and salts, enantiomers, analogs, esters, amides, prodrugs, active metabolites, and derivatives thereof; 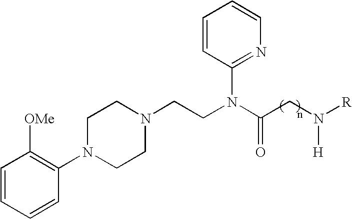
mm. Piperidines and pyrrolidines (illustrated below) as disclosed in PCT Publication No. WO 97/40038 and salts, enantiomers, analogs, esters, amides, prodrugs, active metabolites, and derivatives thereof; 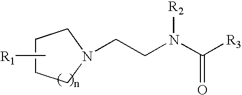
nn. The compound (+)-MCU-629 as shown below and salts, enantiomers, analogs, esters, amides, prodrugs, active metabolites, and derivatives thereof; 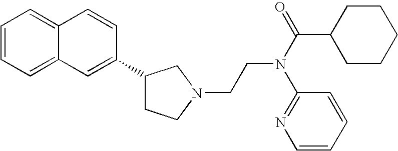
oo. Benzoxazinone derivatives (illustrated below) as disclosed in PCT Publication No. WO 03/091248 and salts, enantiomers, analogs, esters, amides, prodrugs, active metabolites, and derivatives thereof; 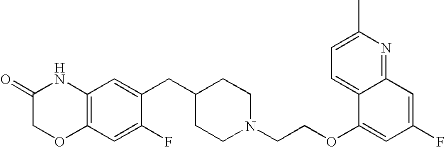
pp. Indole derivatives (illustrated below) as disclosed in PCT Publication WO 01/46181 and salts, enantiomers, analogs, esters, amides, prodrugs, active metabolites, and derivatives thereof; 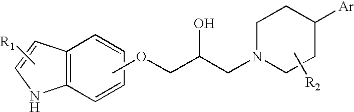
qq. The compound shown below and salts, enantiomers, analogs, esters, amides, prodrugs, active metabolites, and derivatives thereof; 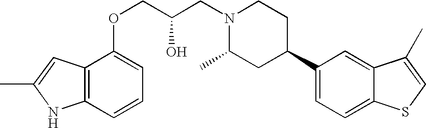
rr. Tetrahydropyridine and piperazine derivatives (illustrated below) as disclosed in U.S. Pat. Nos. 6,596,722, 6,476,035, and 6,391,882, U.S. Patent Application Nos. 2002/0035113, 2002/0173512, and 2003/0018050, and PCT Publication Nos. WO 00/43382, WO 99/05140, and WO 99/67237 and salts, enantiomers, analogs, esters, amides, prodrugs, active metabolites, and derivatives thereof; and 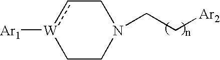
ss. The compound LU-36-274 as shown below and salts, enantiomers, analogs, esters, amides, prodrugs, active metabolites, and derivatives thereof.

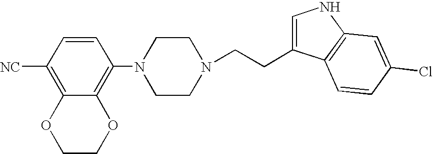

41. The packaged kit of claim 40, wherein the sexual dysfunction is Premature Ejaculation.

42. The packaged kit of claim 40, wherein the active agent is a compound selected from the group consisting of:

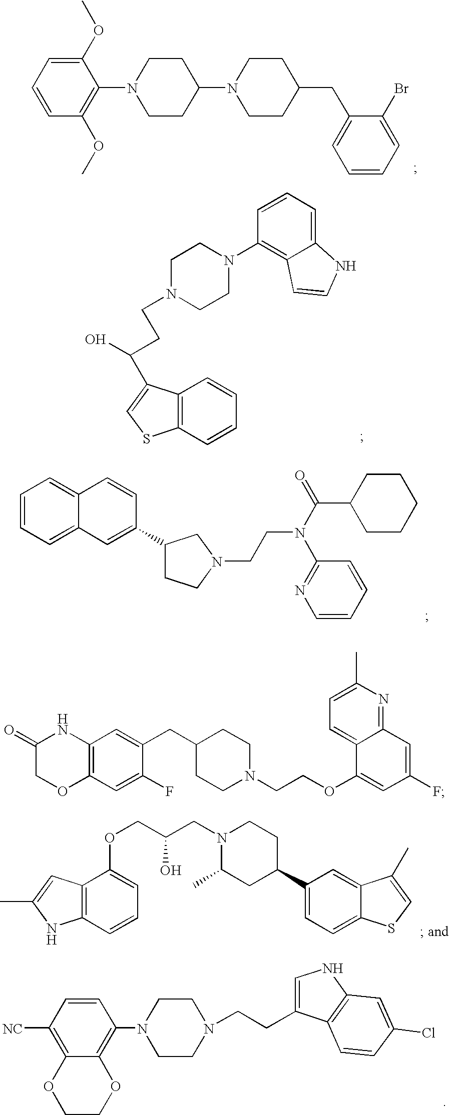

43. The packaged kit of claim 40, wherein the active agent comprises the following compound [chemical structure]
